(19) United States
(12) Patent Application Publication
(10) Pub. No.: US 2019/0225930 A1
(43) Pub. Date: Jul. 25, 2019

(54) MICROFLUIDIC FILTER DEVICES AND METHODS

(71) Applicant: HysOcean, Inc., San Diego, CA (US)

(72) Inventors: Fanqing Chen, Moraga, CA (US); Robert P. Chebi, San Carlos, CA (US); Binbin Fang, Hayward, CA (US)

(21) Appl. No.: 16/332,129

(22) PCT Filed: Sep. 11, 2017
(86) PCT No.: PCT/US2017/050976

§ 371 Date: Mar. 11, 2019

Related U.S. Application Data
(60) Provisional application No. 62/394,112, filed on Sep. 13, 2016.

(51) Int. Cl. C12M 1/00; C12M 1/26; B01L 3/00; G01N 1/40; B01D 39/14; B01D 29/03

(52) U.S. Cl. CPC C12M 47/04; C12M 33/14; B01L 3/502753; G01N 1/4077; B01D 39/14; B01D 29/03; B01L 2300/0681; B01L 2300/12; B01L 2300/123; B01L 2300/0858; B01L 2300/0809; G01N 2001/4088; B01L 2300/08

(57) ABSTRACT

Microfluidic devices for capturing objects that may be, for example, a red blood cell. The device can include at least one filter that includes a filter structure comprising multiple through holes from a first side of the filter structure to a second side of the filter structure and arranged in a known repeating pattern, each of the through holes having a first opening on the first side of the filter structure, a second opening on the second side of the filter structure, and a passageway through the filter structure between the first and second openings. The filter structure may have a thickness of 1 μm to 20 μm, and a substrate including a plurality of vanes that supports at least a portion of the filter structure, the filter structure disposed relative to the plurality of vanes such that the second side of the filter structure is adjacent to the plurality of vanes.

### CROSS REFERENCE TO RELATED APPLICATION

This application claims the benefit of U.S. Provisional Application No. 62/394,112, filed Sep. 13, 2016, which is hereby incorporated by reference in its entirety.

### BACKGROUND

### Technical Field

The embodiments disclosed herein relate to methods and devices for isolating, analyzing, manipulating, and extracting objects of interest such as cells or microbeads using a microfluidic hydrodynamic trap and filter structure in a microfluidic chip.

### Description of the Related Art

Isolation of cells of interest from cell samples containing both cells of interest and cells not of interest for non-invasive diagnosis presents various challenges. For example isolating circulating fetal cells (CFC) from maternal blood containing other maternal and fetal cells not of interest for non-invasive prenatal diagnosis presents challenges due to the rarity of fetal red cells in the maternal blood. The same problem exists in isolation of rare circulating tumor cells (CTC) from blood for liquid biopsies. In these situations, various approaches have been attempted to extract and analyze cells of interest for downstream genetic analysis and diagnostic assays, but the success and purity of extraction has been very poor. Additionally, throughput of such detection and extraction systems remains low, presenting another challenge in the field of non-invasive testing. For example, some methods of isolating cells of interest utilize cell samples plated or spread on a slide or plate for analyzing, isolating, and extracting cells for further analysis. However, the spreading methods employed present challenges because cells often clump together in more than one layer and overlap with each other, making it very difficult to identify boundaries of each cell to determine if the cell is a cell of interest. Imaging the cells from both the top and the bottom of the slide or plate is also not feasible, thus limiting the quality of analysis that can be performed on the cells. Further, imaging cytometry methods currently rely on high resolution imaging in order to analyze all material, not just cells of interest, that are spread on the slide or plate. But, high resolution imaging is memory intensive and uses expensive equipment in a slow, labor intensive process, and further fails to account for precisely identifying individual cells of interest in an efficient and highly accurate manner.

### SUMMARY

Some of the present embodiments may include a multi-layer microfluidic device configured to capture and isolate cells of interest using morphology-based isolation. In some aspects, the multi-layer microfluidic device may include a first layer comprising a microfluidic filter structure, such as a microfluidic filter material or microfluidic filter membrane, disposed on a second layer comprising a support structure, such as a substrate. For example, the filter membrane may be deposited as a thin film onto the substrate or the filter membrane may be spun onto the substrate. A microfluidic chip can include one or more of the multi-layer microfluidic devices. Although the above-described embodiment is a dual-layer microfluidic device, other embodiments are possible. For example, multi-layer microfluidic devices described herein can include a microfluidic filter structure that includes 1, 2, 3, or more layers. For another example, multi-layer microfluidic devices described herein can include a support structure having one or more layers.

Embodiments described herein can include at least one microfluidic filter structure configured to isolate cells of interest from a sample containing cells of interest while simultaneously positioning the cell in a distinct, precisely-defined location of the filter structure that is spatially separate from other distinct, precisely-defined locations of the filter structure. Embodiments of microfluidic devices described herein include a filter structure, such as a microfluidic filter material or microfluidic filter membrane, that automatically creates a monolayer of cells of interest as a stained sample flows over or through the microfluidic device. In some aspects, the filter structure includes a filter membrane comprising multiple through holes specifically shaped and dimensioned to capture cells of interest while permitting cells not of interest to pass through the through holes in the filter membrane and thereby remain uncaptured. The through holes are specifically arranged in a predetermined and repeating grid-like pattern.

Through holes described herein include a first opening on a first side of a filter membrane, a second opening on a second, opposing side of the filter membrane, and a passageway through the filter membrane between the first and second openings. The passageway can include one or more sidewalls within the interior of the filter membrane. Through holes described herein allow objects to translocate through a filter membrane. For example, through holes can allow an object initially present on one side of the filter membrane to translocate through the membrane to a region on the opposing side of the membrane. In some cases, through holes do not allow an object of interest to pass through the membrane, and retain the object of interest on one side of the membrane. Objects retained in this manner can create a monolayer of objects of interest on one side of the filter membrane.

The shape of the opening of a through hole formed in filter membranes described herein can vary. As will be described in detail below, the opening of a through hole on a first side of the filter membrane can have a circular shape. Other shapes are possible. For example, in some implementations the filter membrane includes through holes with openings that are generally rectangular in shape. As will be described in detail below, openings having a rectangular shape can advantageously facilitate flow of the sample through the filter membrane and capture of objects of interest in the filter membrane. Additionally, openings of through holes described herein may also include chamfered or rounded corners that advantageously facilitate the smooth flow of a sample containing cells of interest through the through holes. In one non-limiting example, an opening of a through hole in a first side of a filter membrane has a generally rectangular shape with four corners or edges, and one or more of the corners are chamfered or rounded. The opening of the through hole in the second, opposing side of the filter membrane may also have a generally rectangular shape, and may or may not include chamfered or rounded corners.

Embodiments of filter membranes described herein can include through holes with passageways or sidewalls that are generally perpendicular to the first and second sides of the filter membranes. In other embodiments of filter membranes described herein, through holes have tapered sidewalls that extend through the interior of the filter structure between a first and second side of the filter membrane at an angle. In one non-limiting aspect, a through hole includes a sidewall that is tapered at an angle relative to a line that is generally perpendicular to the first and second side of the filter membrane. Further, filter membranes described herein may be constructed or formed of a material that is mechanically and chemically stable, chemically and electronically inert, hydrophilic and transparent in at least the visual spectrum of light. In some aspects, the support substrate may further comprise support vanes formed out of or into the substrate material. The support vanes can be configured to provide structural integrity to a filter membrane disposed adjacent to the support substrate, and may define a shape and size of one filter region in the filter membrane. In embodiments where portions of a filter membrane are suspended over, but are not in direct contact with, a support substrate, the support vanes can provide structural integrity to the portions of the filter membrane that are suspended over the support substrate. In some embodiments, vanes in support structures described herein may also define a field-of-view (“FOV”) of an imaging cytometry process, where the shape and size of the vane-defined FOV generally matches the shape and size of one filter region in filter membrane.

One innovation includes a device including at least one filter having a filter structure comprising multiple through holes from a first side of the filter structure to a second side of the filter structure and arranged in a repeating pattern such that each of the through holes is separated from the other through holes by a horizontal pitch and a vertical pitch, each of the through holes having a first opening on the first side of the filter structure, a second opening on the second side of the filter structure, and a passageway through the filter structure between the first opening and the second opening, the passageways of the through holes including one or more sidewalls extending between the first opening to the second opening, the first and second openings sized to capture an object in the through hole, wherein the filter structure has a thickness greater or equal to 1 μm and less than or equal to 20 μm measured along a z-axis of the filter structure. The device also includes a substrate having a plurality of vanes that supports at least a portion of the filter structure, the filter structure disposed relative to the plurality of vanes such that the second side of the filter structure is adjacent to the plurality of vanes.

Various implementations can include one or more of the following other aspects and features. For example, the device can include a microfluidic chip having a plurality of filters. The plurality of filters can be arranged in a grid-like pattern. The substrate can include one or more of polydimethylsiloxane (PDMS), glass, polymethylmethacrylate (PMMA), polyethylene terephthalate (PET) or polycarbonate (PC). In some implementations, the filter structure may include one or more of polydimethylsiloxane (PDMS), glass, polymethylmethacrylate (PMMA), polyethylene terephthalate (PET) or polycarbonate (PC). In some implementations, the filter structure comprises silicon oxynitride. In some implementations, a size of the first opening size is different than a size of the second opening. In some implementations, the passageways of the through holes include one or more sidewalls extending between the first opening to the second opening. In some implementations, the one or more sidewalls include at least one tapered sidewall. The at least one tapered sidewall is tapered at an angle relative to a line that is generally perpendicular to the first and second side of the filter membrane. In some implementations, the at least one tapered sidewall has two or more regions, each of the two or more regions tapered at a different angle relative to a line that is generally perpendicular to the first and second side of the filter membrane. In some implementations, the at least one tapered sidewall includes three regions, each of the three regions tapered at a different angle relative to a line that is generally perpendicular to the first and second side of the filter membrane. In some implementations, the one or more sidewalls are curved. In some implementations, the first and second openings have a first dimension of between about 4 μm and about 10 μm and a second dimension of between about 4 μm and about 10 μm. In some implementations, the through holes are dimensioned to capture and retain a single red blood cell in the through hole. In some implementations, the through holes are dimensioned to allow a mature disk-shaped red blood cell to pass through the through hole and capture a single fetal nucleated red blood cell in the through hole. In some implementations, the through holes are rectangular shaped. In some implementations, the first openings of the through holes have one or more corners that are chamfered or rounded. In some implementations, the second openings of the through holes have one or more corners that are chamfered or rounded. In some implementations, the through holes are oval-shaped. In some implementations, the through holes are circular-shaped. In some implementations, a cross-section of the second opening has at least one dimension that is smaller than one dimension of a cross-section of the first opening. In some implementations, the first openings and second openings each have a first dimension of between about 4 μm and about 10 μm and a second dimension of between about 4 μm and about 10 μm. In some implementations, the through holes have generally circular openings with diameters between about 4 μm and about 10 μm. In some implementations, the horizontal pitch is about 20 μm and the vertical pitch is about 10 μm. In some implementations, the plurality of vanes are hexagonal-shaped. In some implementations, The plurality of vanes are rectangular-shaped. In some implementations, the plurality of vanes are square-shaped. In some implementations, the vanes of a thickness of about 0.1 millimeter. In some implementations, the filter structure is formed on the substrate. In some implementations, the filter structure has a thickness in the range of about 1 μm to about 20 μm. In some implementations, the through holes are dimensioned to capture and retain a single red blood cell in the through hole. In some implementations, the through holes are dimensioned to allow a mature disk-shaped red blood cell to pass through the through hole and capture a single fetal nucleated red blood cell in the through hole. In some implementations, the filter materials are configured to withstand pressures greater than or equal to 3 psi resulting from fluid flow over and/or though the filter structure.

Another innovation includes a device including at least one filter, having means for capturing cell-sized objects each in one of a plurality of through holes arranged in a repeating pattern, and means for supporting the means for capturing, where the at least one filter is configured to withstand pressures greater than or equal to 3 psi resulting from fluid flow over and/or though the at least one filter. Such a device may include one or more other features or aspects. For example, the means for capturing may include a filter structure having multiple through holes from a first side of the filter structure to a second side of the filter structure and arranged in a repeating pattern such that each of the through holes is separated from the other through holes by a horizontal pitch and a vertical pitch, each of the through holes having a first opening on the first side of the filter structure, a second opening on the second side of the filter structure, and a passageway through the filter structure between the first opening and the second opening, the passageways of the through holes including one or more sidewalls extending between the first opening to the second opening, the first and second openings sized to capture an object in the through hole, and where the means for supporting includes a substrate including a plurality of vanes that supports at least a portion of the filter structure, the filter structure disposed relative to the plurality of vanes such that the second side of the filter structure is adjacent to the plurality of vanes. In some implementations, the one or more sidewalls include at least one tapered sidewall. In some implementations, the at least one tapered sidewall is tapered at an angle relative to a line that is generally perpendicular to the first and second side of the filter membrane. In some implementations, the at least one tapered sidewall has two or more regions, each of the two or more regions tapered at a different angle relative to a line that is generally perpendicular to the first and second side of the filter membrane. In some implementations, the at least one tapered sidewall includes three regions, each of the three regions tapered at a different angle relative to a line that is generally perpendicular to the first and second side of the filter membrane. In some implementations, the one or more sidewalls are curved. In some implementations, the through holes are dimensioned to capture and retain a single red blood cell in the through hole. In some implementations, the through holes are dimensioned to allow a mature disk-shaped red blood cell to pass through the through hole and capture a single fetal nucleated red blood cell in the through hole.

### BRIEF DESCRIPTION OF THE DRAWINGS

The disclosed aspects will hereinafter be described in conjunction with the appended drawings, provided to illustrate and not to limit the disclosed aspects, wherein like designations denote like elements.

FIG. 1A illustrates a perspective view of a first side of one embodiment of a microfluidic device for capturing and positioning cells of interest according to the present disclosure.

FIG. 1B illustrates a perspective view of a second, opposing side of the microfluidic device illustrated in FIG. 1A.

FIG. 1C illustrates a perspective view of a first side of another embodiment of a microfluidic device for capturing and positioning cells of interest according to the present disclosure.

FIG. 2 illustrates another embodiment of a microfluidic device for capturing and positioning cells of interest according to the present disclosure.

FIGS. 3A, 3B, and 3C illustrate various embodiments of microfluidic devices for capturing and positioning cells of interest according to the present disclosure.

FIG. 4 illustrates a portion of one embodiment of a filter membrane having rectangular through holes according to the present disclosure.

FIG. 5A is an image of a portion of a filter membrane having oval-shaped through holes according to the present disclosure.

FIGS. 5B, 5C, 5D, and 5E are close-up images of a single through hole of the filter membrane illustrated in FIG. 5A.

FIGS. 6A, 6B, 6C, 6D, and 6E are sectional side views of a single through hole of the filter membrane illustrated in FIG. 5A.

FIGS. 7A, 7B, and 7C illustrate various embodiments of microfluidic devices for capturing and positioning cells of interest according to the present disclosure.

FIG. 8A is an image of a portion of a filter membrane having circular-shaped through holes according to the present disclosure.

FIGS. 8B and 8C are close-up images of a single through hole of the filter membrane illustrated in FIG. 8A.

FIG. 9A is an image of a portion of another filter membrane having circular-shaped through holes according to the present disclosure.

FIGS. 9B and 9C are close-up images of a single through hole of the filter membrane illustrated in FIG. 9A.

FIGS. 10A and 10B illustrate an embodiment of a microfluidic device for capturing and positioning microbeads of interest according to the present disclosure.

FIGS. 11A and 11B illustrate another embodiment of a microfluidic device for capturing and positioning microbeads of interest according to the present disclosure.

FIG. 12 is an example flow diagram illustrating one method capturing, isolating, analyzing, and harvesting cells of interest using a microfluidic device according to the present disclosure.

FIG. 13 illustrates an example image taken of an embodiment of a microfluidic chip for capturing and imaging cells of interest according to the present disclosure.

FIGS. 14A and 14B illustrate example images taken of an embodiment of a microfluidic chip for capturing and imaging cells of interest according to the present disclosure.

FIGS. 15A through 16B illustrate processing steps of an example process for fabricating a microfluidic chip for capturing and imaging cells of interest according to the present disclosure.

### DETAILED DESCRIPTION OF CERTAIN ILLUSTRATIVE EMBODIMENTS

Embodiments of methods and devices disclosed herein can obtain cells of interest using morphology-based isolation. Methods and devices disclosed herein advantageously use a filter device integrated in a microfluidic device, such as a microfluidic chip. Although filter devices described herein can be a “filter,” a “lattice,” a “filter platform,” a “filter grid,” a “filter structure,” a “filter material,” a “filter membrane,” or other structures that can filter objects of interest from a sample, filter devices will be referred to as “a filter membrane” throughout this disclosure. In one non-limiting example, a filter membrane isolates cells of interest from a sample containing cells of interest and other objects that are not of interest, such as cells that are not of interest. Isolating cells of interest can include capturing a cell in a filter membrane while simultaneously positioning the cell in a distinct, precisely-defined location of the filter membrane that is spatially separated from other distinct, precisely-defined locations of the filter membrane. The sample may contain non-cellular matter and/or cells that are not of interest, in addition to cells of interest. Embodiments of the filter membranes described herein capture some, most, or all of the cells of interest, such that the cells of interest may be isolated from samples containing numerous cells, at least some of which may be cells not of interest. In some embodiments, the filter membrane can also be used in imaging devices and cytometry processes to detect the precise location of cells that have been captured in the filter membrane, assess the characteristics of captured cells to determine if they are cells of interest, and harvest or pluck cells that have been determined to be of interest for downstream analysis, such as genetic and/or diagnostic analysis.

Filter membranes described herein may comprise multiple through holes specifically shaped and dimensioned to capture cells of interest while permitting cells not of interest to pass through the through holes in the filter membrane and thereby remain uncaptured. Filter membranes described herein can also capture objects of interest that are not cells. For example, filter membranes can capture microbeads of interest in a sample including microbeads that are of interest and microbeads that are not of interest. It will be understood that filter membranes described herein are not limited to capture cells and microbeads, however, such that filter membranes can capture other types of objects contained in a sample having objects of interest with physical characteristics (for example, morphology, size, etc.) that differ from physical characteristics of objects that are not of interest.

Although features of the filter membrane that capture some cells while allowing other cells to pass through the filter membrane can be referred to as pores, wells, recesses, filter holes, through holes, hydrodynamic traps, or other terms, these capturing features will be referred to as “through holes” throughout this disclosure. For example, methods and devices disclosed herein may be used for fetal cell sorting and isolation from maternal blood samples for non-invasive prenatal diagnosis. In one aspect, methods and devices disclosed herein isolate and analyze such cells for downstream genetic analysis and diagnostic assays.

In one non-limiting example, the filter membrane is a morphology-based selection filter that separates cells of interest (such as fetal nucleated red blood cells (“fnRBCs”)) based on two criteria: morphology and biomarkers specific to the cells of interest. Embodiments of filter membranes described herein can separate, or filter, fetal nucleated red blood cells (fnRBCs) from a maternal blood sample containing mature (non-nucleated) maternal RBCs and fetal nucleated RBCs. Fetal nucleated RBCs circulating in the maternal blood are extremely rare, with some estimates as low as 1 in a 10 million. Mature human RBCs are oval biconcave disks and generally lack a cell nucleus. In contrast, fetal nucleated RBCs are slightly larger than mature maternal RBCs and generally spherical rather than disk-shaped. Embodiments of the morphology-based selection filters described herein include through holes with a specific shape, size, and arrangement such that most or all of the mature red blood cells (RBCs) in a sample pass through the through holes in the filter while some, most, or all of the fetal nucleated RBCs are retained or “captured” in the through holes. However, due to variations in the morphology of the RBCs, some maternal RBCs may also be captured in through holes in the filter even though they are not cells of interest.

In one implementation, a filter membrane according to the present disclosure includes a first side, a second, opposing side, and multiple through holes passing through the filter membrane from the first side to the second side. The through holes are arranged in a precisely-defined grid-like pattern in the filter membrane. Each through hole includes a first opening in the first side of the filter membrane, a second opening in the second side of the filter membrane, and a passageway through the filter membrane extending from the first opening to the second opening, such that objects initially present near the first side of the filter membrane can translocate through the passageway to a region near the opposing, second side of the filter membrane. In this implementation, the first opening and the second opening of each through hole are generally circular in shape, with a diameter of about 7 microns. In this non-limiting example, each through hole in the filter membrane is specifically shaped (for example, a first opening having a circular shape) and dimensioned (for example, 7 micron diameter) to capture a cell of interest (for example, a circular cell from a cancerous tumor that generally has a 10 micron diameter) in the one single through hole, while permitting a cell that is not of interest (for example, a cell that is not from the cancerous tumor and has a diameter that is generally less than 7 microns) to pass through a first opening and out a second opening of a through hole, thereby remaining uncaptured in the filter membrane. In some aspects, each cell of interest (for example, each tumor cell having a generally circular morphology and a 7 micron diameter) is captured in one single through hole of the filter membrane, such that each of the captured tumor cells together create a monolayer of cells of interest on the first side of the filter membrane.

In some embodiments, the filter is made of any suitable material that provides optimal transparency characteristics and the optimal strength and physical properties for the intended cell capturing application. For example, in some embodiments, the filter membrane includes a material or materials that are transparent to light in the visual spectrum (e.g., wavelengths of approximately 400 nanometers to approximately 700 nanometers). In some embodiments, the filter material is transparent to light beyond the visible spectrum, including, but not limited to, light having wavelengths in the near infra-red (NIR) and near ultra-violet (NUV) spectrums. One non-limiting advantage of a filter membrane including a transparent material or materials is that cells captured in the filter membrane can be optically imaged from multiple directions. For example, in one implementation, a multi-layer microfluidic device includes a filter structure, such as a filter membrane described herein, disposed on top of, disposed adjacent to, or suspended over a support substrate. Embodiments of filter membranes that are transparent to light allow the microfluidic device to be imaged from either of two directions: (1) from a “top” of the microfluidic device (for example, from the side of the device closest to filter membrane); or (2) from a “bottom” of the microfluidic device (for example, from the side of the device closest to the support substrate). In some embodiments, the use of a transparent material further facilitates imaging of captured cells from the top of the microfluidic device and/or from the bottom of the microfluidic device. In contrast, conventional microfluidic devices include filter structures that can only be imaged from one direction (for example, only from the top of the device) with an epifluorescence using bright field illumination.

Embodiments of microfluidic devices described herein can advantageously include a filter membrane formed of a material or materials that withstand pressure exerted on the filter membrane as a fluid sample flows through the microfluidic device. Additionally, the filter membrane can include a material or materials selected to have specific mechanical properties to withstand pressures exerted by a micromanipulator harvesting, removing, and/or plucking cells of interest or cells not of interest from the filter membrane, without tearing, breaking, or degrading the filter membrane. In some aspects, a single fluid sample can be run repeatedly through the same filter membrane, or different portions of a single fluid sample can be run sequentially through the same filter membrane, without tearing, breaking, or degrading the filter membrane. This allows for cells of interest to be harvested from the filter membrane between each run, after a subset runs, or after all runs are complete, but in all cases the same filter membrane is advantageously used for all runs without replacing the filter membrane or providing a system having multiple filter membranes to process a single sample.

In some embodiments, the filter membrane includes a material or materials that do not fluoresce under illumination by a light source and/or suppress background fluorescence. In some embodiments, following and/or during capture and isolation of cells in the filter membrane, the cells may be labeled or stained with nuclear stains, biomarkers, and/or fluorescent dyes. Such fluorescent molecules or dyes may produce a corresponding light signature or spectrum upon being illuminated or excited by a light source having a particularly corresponding wavelength of light. Thus, using specific fluorescent molecules or dyes may produce an indicator of a particular nucleic acid, antibody or antibody-based fragment probe present on or within a captured cell, which may be imaged using a microscope or other imaging platform. One non-limiting advantage of suppressing or negating the background fluorescent of the filter membrane itself is that total background fluorescence is kept low to avoid interfering with imaging of fluorescent- or light-based indicators of captured cells during imaging processes such as imaging cytometry.

In another aspect described in detail below, embodiments of microfluidic devices including filters described herein represent a capture platform having a specifically arranged, predetermined, and repeating grid-like pattern of through holes. In cases where the capture platform (such as a microfluidic device) includes a plurality of filter membranes, the location of each filter membrane in the capture platform can be precisely determined. Additionally, the precise location of each through hole in each filter membrane can be very accurately determined, down to the nanometer range in some aspects.

Additionally, embodiments of filter membranes described herein capture and position cells in a way that increases the speed at which the captured cells can be analyzed and harvested. For example, captured cells may be identified and/or located based on information about the position or location of a cell in the filter membrane (for example, a precise x, y coordinate on the filter membrane). Accordingly, a captured cell of interest can be verified as a cell of interest (for example, a fetal nucleated RBC or fetal trophoblast) and the filter membranes for capturing and positioning cells described herein can increase the speed at which cells of interest are removed from the filter membrane, or “harvested,” for downstream testing.

One non-limiting advantage of methods and devices disclosed herein includes, but is not limited to, the ability to automatically create a monolayer of cells as a stained sample flows through a microfluidic filter membrane. Embodiments of microfluidic devices described herein may be configured to create a monolayer of cells by preventing one potential cell of interest of the sample from sitting or lying on top of another potential cell of interest, which can cause imaging and resolution complications due to the imaging procedure needing to distinguish the two closely-spaced cells from one another. Implementations of the microfluidic filter membranes disclosed herein can include a layer of material having through holes that pass entirely through the layer of material from one side of the material to the other, opposing side of the material such that an object in a sample flowing through the filter membrane can translocate through the layer of material. In some cases, the filter membrane includes a single layer of material having through holes and in other cases, the filter membrane includes a plurality of layers, where each through hole passes entirely through each of the plurality of layers. Each through hole in the filter membrane is spatially separated from other through holes and configured to capture only a single object of interest, such as a single cell of interest. Thus, each through hole that captures a cell contains a single, isolated cell whose characteristics (such as size, morphology, biomarkers) can be readily distinguished because the cell is spatially separated from other cells that are captured in the filter membrane, and is held at a distinct, precisely-defined location for further analysis. Additionally, embodiments of methods and devices herein can advantageously maximize the density of cells of interest on a single microfluidic filter membrane.

In some embodiments, the filter membrane (or a surface of the filter membrane upon which a sample is introduced) includes a material that is hydrophilic as opposed to hydrophobic. Hydrophilic properties can advantageously permit the fluid sample to flow smoothly through the through holes. Hydrophobic materials, in contrast, may cause the cells in the fluid sample to clump together as the sample is introduced onto the filter membrane, thereby requiring more pressure or force to cause the cells to pass through the through holes. In turn, depending on the mechanical properties of the filter, the additional force required to push cells (such as cells that are not of interest) through the filter membrane may cause the filter membrane to tear, bulge, warp, or bend, thereby inhibiting the ability of the microfluidic device to capture cells of interest.

In some embodiments having through holes with rectangular openings, the through holes may have one or more rounded or chamfered corners. The chamfered corner can advantageously remove dead spots in the fluid flow through the through hole that would ordinarily be present in the case of an opening having sharp angular edges and/or corners. These sharp, angular corners may cause the accumulation of fluid and/or cells within or around the corner. One non-limiting advantage of through holes having rounded or chamfered corners is that the through hole may be configured to permit a smooth flow of fluid samples containing cells, microbeads, or other objects through the filter membrane.

In some embodiments, through holes may include a sidewall extending between a first and second side of the filter membrane (which include one layer or more than one layer), thereby allowing an object to translocate through the filter membrane. For example, the sidewall may be tapered at an angle relative to a line that is generally perpendicular to the first and/or second side of the filter membrane. The tapered sidewall may be configured such that the dimensions of the first opening of the through hole on the first side of the filter membrane are different than the dimensions of the second opening on the second side of the filter membrane. In one example, a diameter of a circular first opening of a through hole on the first side of the filter membrane may be greater than the diameter of a circular second opening of the through hole on the second side of the filter membrane. Embodiments of through holes having tapered sidewalls as described herein advantageously improve capture of cells of interest in the filter membrane. For example, embodiments of filter membranes with through holes having tapered sidewalls can capture more of the cells of interest in a sample flowing through the filter membrane, and in some cases can retain captured cells of interest more securely (while additional fluid samples are passed through the filter, for example).

In some embodiments, the sidewall may be tapered with a variable angle relative to a line that is generally perpendicular to the first and/or second side of the filter membrane. For example, the tapered sidewall may have one or more regions, where each region may be tapered at a different angle relative to the line generally perpendicular to the first and/or second side of the filter membrane. In one example, the tapered sidewall may have two regions where a first region is tapered at a first angle relative to the line generally perpendicular to the first side of the filter membrane and a second region tapered at a second angle relative to the line generally perpendicular to the second side of the filter membrane. In some embodiments, there may be 1, 2, 3, 4, or more regions on the tapered sidewall, each region being tapered at a different angle relative to the line generally perpendicular to the first and/or second side of the filter membrane. In some embodiments, one or more regions may be generally parallel with the line that is generally perpendicular to the first and/or second side of the filter membrane. The angle of the tapered sidewalls and a target dimension of the through hole can be independently variable and can be selected to achieve desired effects. For example, a target dimension for a circular-shaped through hole having tapered side walls can be the smallest diameter of the through hole. In another example, a target dimension for a rectangular-shaped through hole can be the smallest dimension of the through hole measured in the x-direction or the y-direction. The target dimension can be selected to enhance the hydrodynamic trapping capability of a specific through hole, while the angle of the tapered sidewalls can be selected to further help capture, hold, and retain objects of interest (for example, cells, beads, etc.) in place.

In one non-limiting embodiment, the features of an angled sidewall of a through hole serve dual functions: one being a physical hydrodynamic trap preventing captured cells or beads of further lateral or directional movement, and the other being a filtering or isolating membrane. If the through holes do not include tapered side walls, the through holes may only serve to prevent certain cells from flowing through the filtering membrane, but would not serve as a hydrodynamic trap, or capturing grid, for cells or beads of targeted size, thus retaining and immobilizing them within, or partially within, the through holes. In one non-limiting example of a circular through hole, the thickness of the membrane and the angle of the through hole side walls determine the capturing and immobilizing characteristics of the filter membrane, and the smallest diameter, at the bottom of the through hole, determines its filtering or isolating properties. Similar effects can be achieved in non-circular through holes by independently selecting the angle of the tapered side wall and the smallest dimension (measured along the x-axis and the y-axis) of the through hole.

Methods and devices disclosed herein may also permit: 1) cell filtration, staining, enrichment (if needed) on one integrated platform minimizing manual labor and intervention; and 2) use of automation to streamline processing of cell samples.

Methods and devices disclosed herein may be described in reference to an exemplary, non-limiting application of non-invasive prenatal testing (NIPT) and the isolation of fetal nucleated RBCs from maternal RBCs. The skilled artisan will understand, however, that the principles and concepts of the methods and devices are broadly applicable to the capture and study of objects of interest from a sample, such as cells, beads, microbeads, and other particles that are subject to filtration with or without fluorescence-based staining. Accordingly, embodiments of the methods and systems described herein can be used in numerous applications, including but not limited to NIPT. For example, methods and devices disclosed herein may be configured for the isolation of microbeads, tumor cells for oncology, or any other pathological condition where cells of one kind can be differentiated from cells of another kind based on size, morphology, nuclear staining, and/or biomarker identification.

### Definitions

Unless defined otherwise, all technical and scientific terms used herein have the same meaning as is commonly understood by one of ordinary skill in the art to which this invention belongs. All patents, applications, published applications and other publications referred to herein are incorporated by reference in their entirety. If a definition set forth in this section is contrary to or otherwise inconsistent with a definition set forth in the patents, applications, published applications and other publications that are herein incorporated by reference, the definition set forth in this section prevails over the definition that is incorporated herein by reference.

As used herein, the singular forms “a,” “an,” and “the” include plural references unless indicated otherwise. For example, “a” filter membrane includes one or more filter membranes.

As used herein, the term “microfluidic device” or “microfluidic chip” generally refers to a device through which materials, particularly fluid borne materials, such as liquids, can be transported, in some embodiments on a micro-scale, and in some embodiments on a nanoscale. Thus, microfluidic chips described herein can include microscale features, nanoscale features, and combinations thereof. The samples delivered on such a device may be fluids alone or fluids with suspended components such as cells and particles.

An exemplary microfluidic chip can include structural or functional features dimensioned on the order of a millimeter-scale or less, which are capable of manipulating a fluid at a flow rate on the order of 5 mL/min or less. Examples of microfluidic chips described herein can be 5 millimeters×5 millimeters in size measured along an x-axis and a y-axis of the microfluidic chip. In another non-limiting example, microfluidic chips described herein can be 8 millimeters by 8 millimeters in size measured along an x-axis and a y-axis of the microfluidic chip. Other sizes are possible. In one example, a microfluidic chip includes a plurality of filters arranged in a grid-like pattern. The plurality of filters may be arranged in a n×m grid-like pattern, where “n” and “m” may be any integer and need not be the same. For example, the microfluidic chip may comprise a plurality of filters arranged in a 5×5 grid-like pattern. In another non-limiting example, the plurality of filters may be arranged in a 4×4 grid-like pattern. Examples of filters described herein can be approximately 0.9 millimeters by 0.9 millimeters in size measured along an x-axis and a y-axis of the filter. In another non-limiting example, the filters described herein can be approximately 1.2 millimeters by 1.2 millimeters measured along an x-axis and a y-axis of the filter. In another non-limiting example, the filters described herein can be approximately 1.2 millimeters by 5.1 millimeters measured along an x-axis and a y-axis of the filter. Other sizes are possible. In embodiments having asymmetrical dimensions, the filters may be arranged in a 4×1 grid-like pattern. Without subscribing to a particular scientific theory, the size and layout of the filters on the microfluidic chip may be designed to reduce bubble formation and manage microfluidic flow of a liquid through the filter, because imaging of the microfluidic chip may be disrupted or degraded by bubbles generated by fluid flow over and through the filters. In another example, a microfluidic chip includes a single filter membrane supported by a substrate that includes vanes, where the vanes define regions of the filter membrane. In some cases, a microfluidic chip includes additional features such as, but not limited to channels, fluid reservoirs, reaction chambers, mixing chambers, separation regions, and supporting structures. In some examples, a channel includes at least one cross-sectional dimension that is in a range of from about 0.1 μm to about 10 millimeters.

A microfluidic chip can exist alone or can be a part of a microfluidic system which, for example and without limitation, can include: pumps and valves for introducing fluids, e.g., samples, reagents, buffers and the like, into the system and/or through the system; detection equipment or systems; data storage systems; and control systems for controlling fluid transport and/or direction within the device, monitoring and controlling environmental conditions to which fluids in the device are subjected, e.g., temperature, pressure, current, and the like using sensors where applicable. The valves and flow in such systems may be pressure or vacuum driven.

A microfluidic chip can be made from any suitable materials, such as PDMS (Polydimethylsiloxane), glass, PMMA (polymethylmethacrylate), PET (polyethylene terephthalate), PC (Polycarbonate), etc., or a combination thereof. The filter integrated in the chip may be made from similar materials or different materials. In example implementations, filters described herein are made from silicon oxynitride, such as but not limited to SiON or SiO₂.

As used herein, the terms “filter” and “filter membrane” refer to a material that separates objects of interest from other objects that are not of interest. In embodiments described herein, a filter membrane separates objects of interest by retaining objects of interest in through holes in the filter membrane, while objects that are not of interest pass through the through holes which are hydrodynamic traps in the filter membrane. The objects of interest can be, but are not limited to, cells, beads, or microbeads. Embodiments of filter membranes described herein can include a single-layer of material, or include multiple layers, such as two, three, or more layers.

As used herein, the term “through hole” refers to an opening or recess extending through a structure, such as a filter membrane. In one example, the structure includes a first side and a second side, and a through hole includes sidewalls that extend entirely through the structure between the first and the second side. Through holes allow objects to translocate through the structure. For example, through holes can allow an object initially present on one side of the structure to translocate through the structure to a region on the opposing side of the structure. In some cases, through holes do not allow an object to pass through the structure, and retain the object on one side of the structure. Objects that do not translocate through a through hole and are retained in the through hole can be positioned partially or entirely within a through hole. Through holes described herein can be specifically shaped and dimensioned to separate objects of interest from other objects that are not of interest. Through holes may also be referred to as pores, hydrodynamic traps, wells, filter holes, or other terms representing a passageway through a filter membrane, however, these features will be referred to as “through holes” throughout this disclosure. In embodiments described herein, the through holes facilitate the separation and retention of objects from objects not of interest. The through holes can be designed to have specific dimensions corresponding to the shape and size of the objects of interest. In this way, single instances of objects of interest (e.g., a single cell) can be captured in a through hole, while permitting objects not of interest to either be passed entirely through the through hole or inhibited from entering (or being retained within) the through hole. As described above, objects of interest can be, but are not limited to, cells, beads, or microbeads. Through holes may be designed in any shape or size, for example they can have generally circular, rectangular, oval, or other cross-sectional shapes. The shape and size of each through hole may be determined based on the objects of interest being captured by the filter membrane.

It is understood that aspects and embodiments of this disclosure include “consisting” and/or “consisting essentially of” aspects and embodiments.

In the following description, specific details are given to provide a thorough understanding of the examples. However, it will be understood by one of ordinary skill in the art that the examples may be practiced without these specific details. For example, electrical components/devices may be shown in block diagrams in order not to obscure the examples in unnecessary detail. In other instances, such components, other structures, and techniques may be shown in detail to further explain the examples.

Other objects, advantages, and features of the present disclosure will become apparent from the following description taken in conjunction with the accompanying drawings.

Integrated Microfluidic Chip with Filter Membrane

Integrated microfluidic chips for non-invasive isolation of cells (such as, but not limited to, fetal nucleated RBCs) are described herein. The integrated microfluidic chips can include a single filter or a plurality of filters. In embodiments of the microfluidic chip that include a single filter, the filter can include a sheet or layer of filter material (“a filter membrane”) supported by a substrate. Filter membranes described herein can include a single sheet or layer of material, or can include a plurality of sheets or layers of material. In embodiments of microfluidic chips that include a plurality of filters, the plurality of filters can be arranged in a grid-like structure. Some embodiments of the microfluidic chips described herein can also include a binding moiety or affinity molecule. For example, in systems designed to capture fetal nucleated RBCs, the system can include a binding moiety or affinity molecule that specifically binds to a cell-specific antigen or a non-fetal cell-specific antigen for positive selection of fetal cells or negative selection of unwanted cells.

In some embodiments, the integrated microfluidic chip may comprise at least one filter membrane that is transparent and visualizable under a microscope. The filter comprises multiple through holes that are arranged in a repeating grid pattern and are configured to capture, retain, and simultaneously position cells of interest in precisely-defined, clearly-distinguishable locations on the filter membrane (each location corresponding to a single through hole). In some embodiments, the through holes are specifically arranged in a regular and repeating grid pattern where each through hole can be precisely located based on a unique, predetermined X, Y coordinate on the filter membrane. In some embodiments, each filter membrane may include several thousand through holes (e.g., 8,000 or more), thus enabling the capture and imaging of several thousand cells.

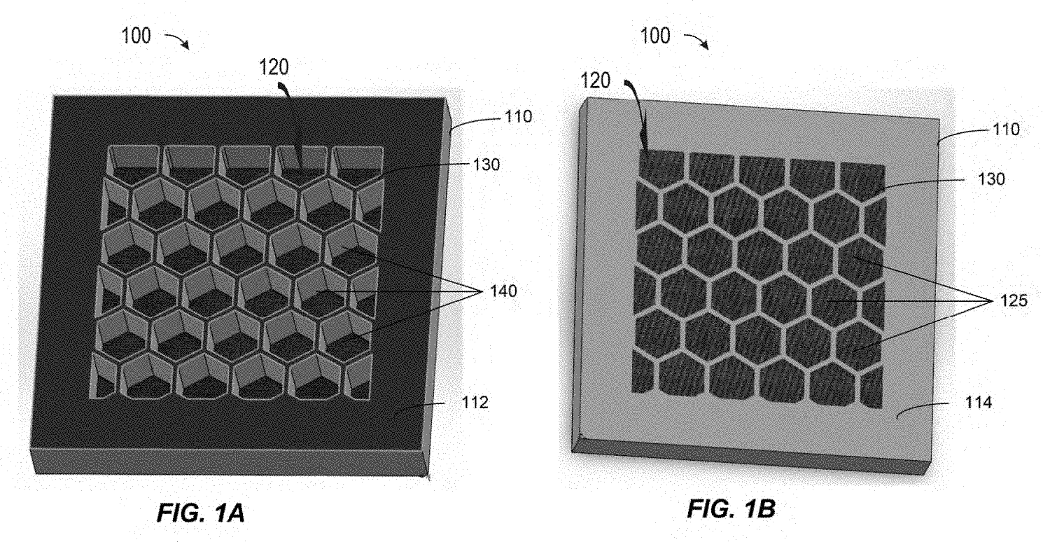

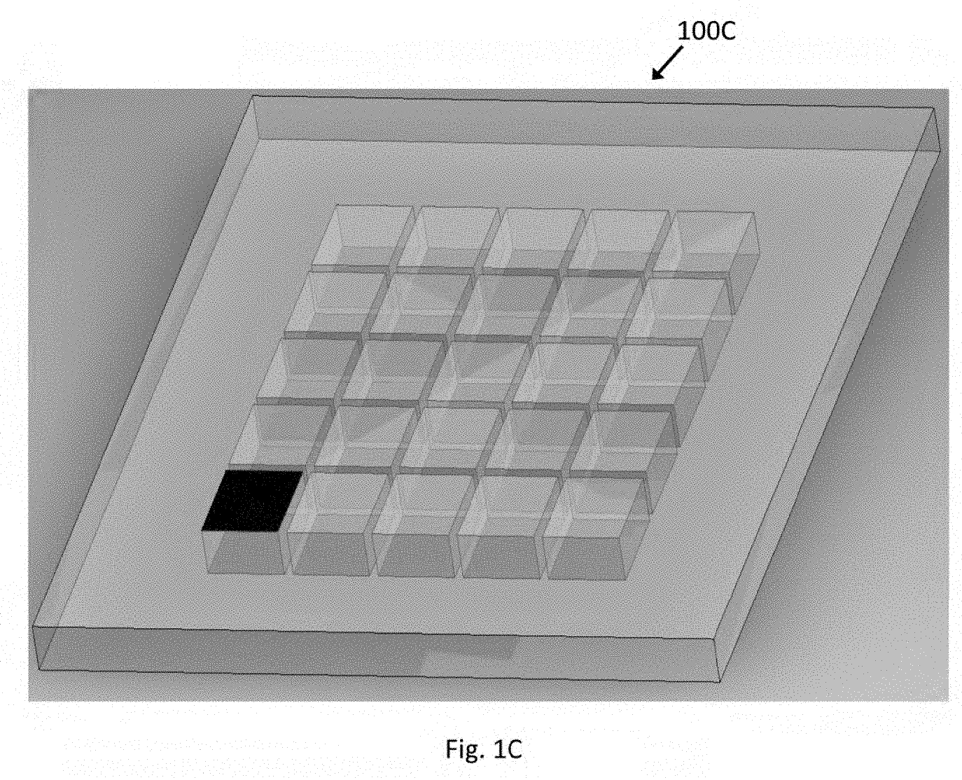

FIGS. 1A and 1B illustrate a first side view and a second side view, respectively, of an exemplary microfluidic chip 100 according to one embodiment. In this non-limiting example, the microfluidic chip 100 is a dual layer structure comprising a support layer and a filter layer. In this case, the support layer includes a substrate 110 and the filter layer includes a filter membrane 120. The substrate 110 includes a first side 112 and an opposing, second side 114. As will be described in detail below, the substrate 110 also includes vanes 130 that extend between the first side 112 and the second side 114. In the illustrated example, the filter membrane 120 is disposed on top of and supported by the side 112 of the substrate 110, and in particular by portions of the vanes 130 located on the side 112. In FIG. 1B, for example, the vanes 130 supporting the filter membrane 120 are visible through the filter membrane 120. The vanes 130 define hexagonal-shaped filter regions 125 of the filter membrane 120. Regions 125 having different shapes are possible (for example, FIG. 1C). In other embodiments (not illustrated in FIG. 1B), a microfluidic chip includes a plurality of hexagonal-shaped filter membranes 120, each filter membrane 120 disposed on or within one hexagonal-shaped region 125 of the substrate 110.

The substrate 110 can be formed of any suitable material and have any suitable dimension to support the filter membrane 120. In some cases, the substrate 110 is a silicon wafer. The silicon wafer can be a commercially available, conventionally-sized wafer that is processed to obtain desired dimensions for the substrate 110. For example, a standard silicon wafer can be thinned down to have a thickness of approximately 400 microns. The thickness of the support material 110 can be selected based on the needs of the particular application for which the microfluidic chip is intended.

The filter membrane 120 can be formed by any suitable means. In one non-limiting aspect, the filter membrane 120 is formed by depositing a very thin layer or layers of material onto the substrate 110. For example, the filter membrane 120 may be deposited as a thin film onto the substrate 110 through plasma enhanced chemical vapor deposition (PECVD) or other thin film deposition techniques.

The filter membrane 120 can be formed to have any suitable thickness for the particular application of the microfluidic chip 100. In some cases, the filter membrane 120 is disposed on, disposed adjacent to, or suspended over a top or bottom surface of the substrate 110 and has a thickness of greater than or equal to 5 microns as measured along a z-axis of the filter membrane. For example, the filter membrane 120 can have a thickness of approximately 20 microns. In other examples, the filter membrane 120 has a thickness of about 1 micron, about 2 microns, about 3 microns, about 4 microns, or about 5 microns as measured along a z-axis of the filter membrane. As will be described in detail below, however, very thin filter membranes such as those described herein are still relatively strong for their thickness and are advantageously rigid enough to withstand pressures associated with a sample fluid flowing through the filter membrane. These characteristics can be particularly beneficial in applications where more than one sample is applied to a single filter membrane, or in applications where a sample must be applied to a filter membrane at relatively high pressure to ensure efficient and accurate capture of cells of interest in the filter membrane.

The filter membrane 120 may be made from similar materials or different materials as the substrate 110. In example implementations, filter membranes described herein include silicon oxynitride, such as but not limited to SiON or SiO₂. However, any material may be suitable that provides the transparency sought and the requested strength and physical properties for the intended cell capturing application. For example, in some embodiments, the filter material 120 is transparent to light in the visual spectrum (e.g., wavelengths from approximately 400 nanometers to approximately 700 nanometers). In some embodiments, the filter material 120 is transparent to light beyond the visible spectrum, including, but not limited to, light having wavelengths in the near infra-red (NIR) and near ultra-violet (NUV) spectrums. One non-limiting advantage of filter membranes including transparent materials is that cells captured in the filter membrane can be imaged from either side of the substrate 110, for example from the first side 112 or the second side 114 of the substrate 110.

In some embodiments, the filter membrane 120 includes a material or materials that do not fluoresce under illumination by a light source and/or that suppresses background fluorescence. In some embodiments, before, during, or after capture and isolation of cells in the filter membrane, the cells may be labeled or stained with nuclear stains, biomarkers, and/or fluorescent dyes. Such fluorescent molecules or dyes may produce a corresponding light signature or spectrum upon being illuminated or excited by a light source having a particularly corresponding wavelength of light. Thus, using specific fluorescent molecules or dyes may produce an indicator of a particular nucleic acid, antibody or antibody-based fragment probe present on or within a captured cell, which may be imaged using a microscope or other imaging platform. One non-limiting advantage of suppressing or negating the background fluorescence of the filter membrane 120 itself is that total background fluorescence is kept low to avoid interfering with imaging of fluorescent- or light-based indicators of captured cells during imaging processes such as imaging cytometry.

In some embodiments, the filter membrane 120 is formed of a material selected to be mechanically and chemically stable as well as chemically and electrically inert. The filter membrane 120 includes a mechanical strength or rigidity to withstand pressure from fluid flow as the cell samples flow over and through the microfluidic chip. Advantageously, the filter membranes 120 described herein have sufficient structural integrity and rigidity to limit or avoid buckling, sagging, or breaking under pressure from the flow of fluid or gravitational forces. For example, filter materials can be selected that withstand pressures greater than or equal to 3 psi resulting from fluid flow over and/or through the filter membrane.

Additionally, the filter membrane 120 can be formed of a material having specific mechanical properties to withstand the insertion of a micromanipulator while harvesting, removing, and/or plucking cells of interest or cells not of interest from the filter membrane. For example, a micromanipulator may include a miniscule needle configured to pluck fragile cells captured in each through hole of the filter membrane. The insertion and removal of the needle in each through hole may exert an outward force onto the sidewalls of a given through hole, thus the filter membrane may be selected to withstand this force such that the filter membrane does not break nor is the through hole deformed.

Additionally, filter membranes with desired holes and configurations could be opaque or translucent for certain applications, made of materials such as silicon, for example. Filter membranes described herein can be generated or formed through specific chemical or electrochemical processes to desired thickness ranging from several microns to tens of microns or possibly hundreds of microns, followed by separative lift-off techniques, and then anodically bonded or attached through specific adhesive techniques to substrates that could be of several materials, or layers of materials, like silicon, organic polymers, glass, or plastics with different shapes and/or sizes.

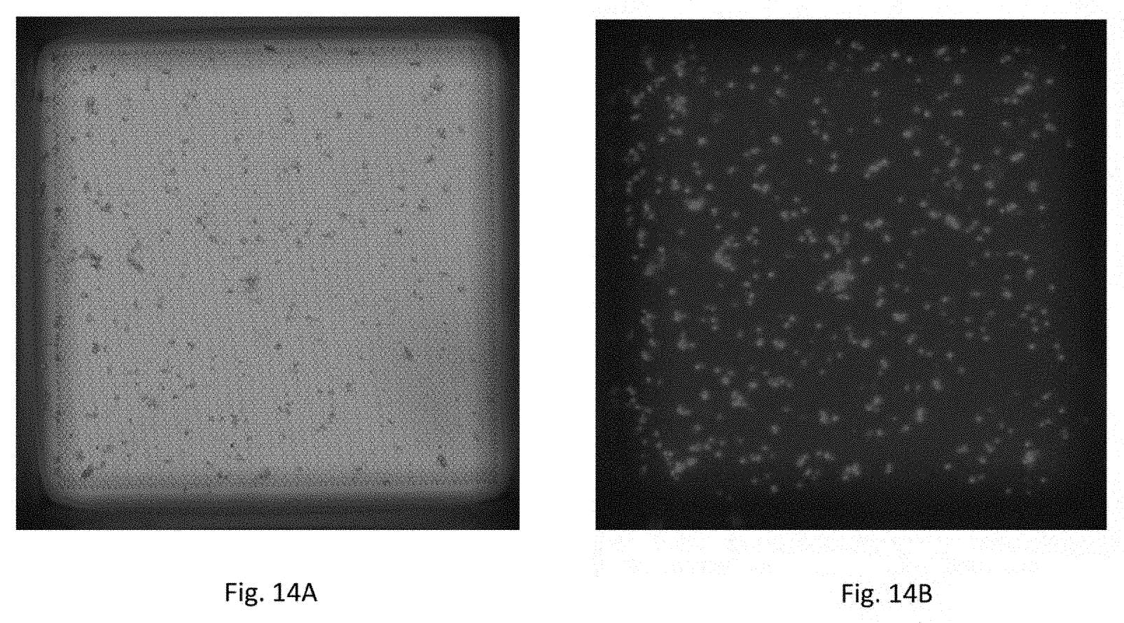

As a result, filter membranes described herein can be used more than once to capture cells of interest in a sample or multiple portions of the same sample, representing a significant improvement over existing filter devices. For example, a first portion of a sample can be applied to the filter membrane 120, capturing a first subset of cells of interest in the first portion of the sample. Subsequently, a second portion of a sample can be applied to the same filter membrane 120, capturing a second subset of cells of interest in through holes of the filter membrane 120 that are not occupied by an object (whether a cell of interest or some other undesired object). This process can be repeated until the entire sample has been applied to the same filter membrane 120, or until it is determined that a sufficient number of cells of interest have been captured in the filter membrane 120. Imaging of the filter membrane 120, manipulation of objects in the filter membrane, or other processes can take place at regular intervals or before the next sample portion is applied to the filter membrane. In some cases, at the end of this capture process, the microfluidic chip 100 will have a very high density of cells of interest in a single filter membrane 120, with each cell of interest isolated in a single through hole at a distinct, precisely-defined x, y location of the filter membrane 120. In one example, a monolayer of cells of interest is held in place on the side 112 of the substrate 110 by the filter membrane 120, and provides a unique platform from which to analyze, identify, and extract cells of interest from the microfluidic chip 100. See, for example, photographs of actual cells of interest held in place on a filter membrane in accordance with embodiments described herein, which are provided in FIGS. 14A and 14B and described in detail below.

In some embodiments, the filter membrane 120 is formed of a material having hydrophilic properties, as opposed to hydrophobic properties. The hydrophilic properties of the filter membrane 120 permit the fluid sample to flow smoothly through the through holes. In some implementations, a surface on the first side of the filter membrane 120 is treated to obtain hydrophilic characteristics. In other implementations, the filter membrane 120 is formed of a material or materials having the desired hydrophilic characteristics. Advantageously, the hydrophilic properties of the filter membrane can prevent cells from clumping together as they flow through the filter membrane 120, thereby reducing the amount of pressure or force that is required to push the sample (and cells that are not of interest) through the through holes of the filter membrane 120. This reduction in the amount of pressure or force exerted on the filter membrane 120 during the capture process represents a significant improvement over existing filter systems, because embodiments of the filter membranes 120 described herein are less likely to puncture, bend, warp, bulge, or otherwise degrade during one or more capture processes, resulting in a longer life span of a single filter membrane 120 and the ability to use a single filter membrane 120 for multiple capture processes.

In some embodiments, the substrate 110 of an exemplary microfluidic chip 100 typically employs a solid or semi-solid substrate that may be planar in structure, i.e., substantially flat or having at least one flat surface. Suitable substrates may be fabricated from any one of a variety of materials, or combinations of materials. Often, the planar substrates are manufactured using solid substrates common in the fields of microfabrication, e.g., silica-based substrates, such as glass, quartz, silicon or polysilicon, as well as other known substrates, i.e., gallium arsenide, to ensure superior manufacturability and enhanced repeatedly of target dimensions. In the case of these substrates, common microfabrication techniques, such as photolithographic techniques, wet chemical etching, micromachining, i.e., drilling, milling, and plasma etching, and the like, may be readily applied in the fabrication of microfluidic chips and substrates. Alternatively, polymeric substrate materials may be used to fabricate the devices of the present disclosure, including, for example, polydimethylsiloxanes (PDMS), polymethylmethacrylate (PMMA), polyurethane, polyvinylchloride (PVC), polystyrene, polysulfone, polycarbonate, and the like. In the case of such polymeric materials, injection molding or embossing methods may be used to form the substrates. In such cases, original molds may be fabricated using any of the above described materials and methods. The assembled microfluidic chips may be treated with plasma to alter surface wet-ability where desired post assembly or preferably treated first and then assembled.

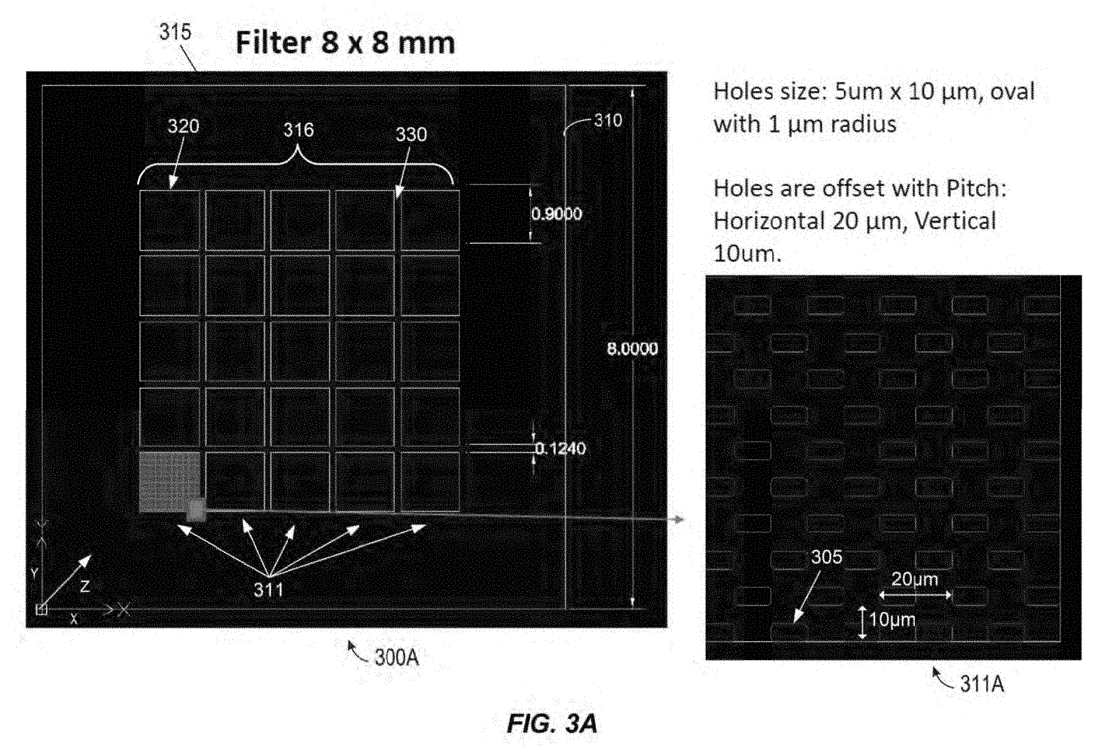

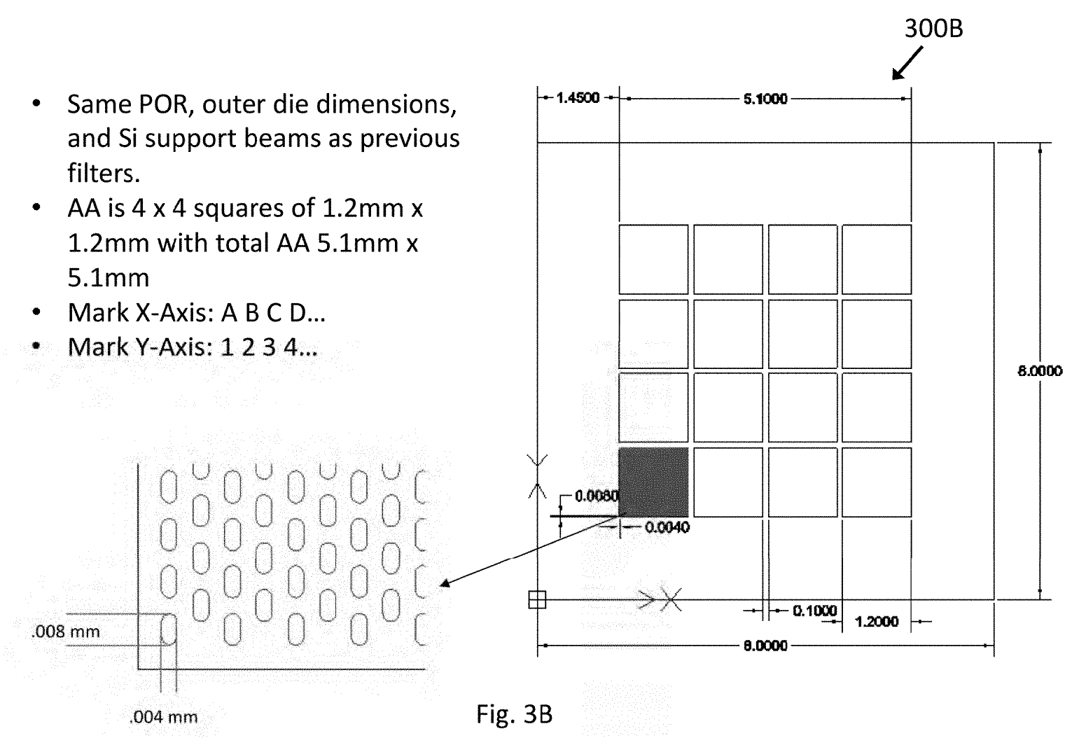

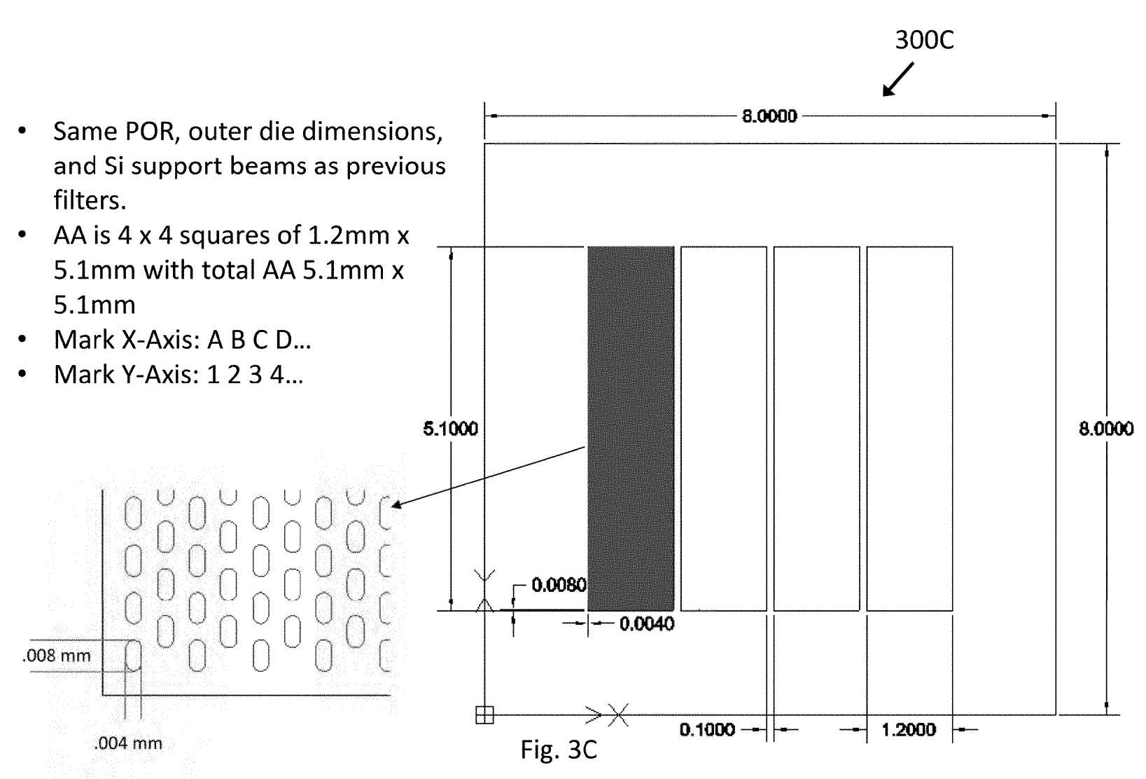

The substrate 110 can be formed or manufactured to include multiple support vanes 130. For example, common microfabrication techniques and/or injection molding or embossing methods can be applied in the fabrication of the substrate 110 to include vanes 130. In some embodiments (not illustrated), individual filter membranes 120 are held or disposed within hexagonal-shaped regions defined by vanes 130. In the embodiment illustrated in FIGS. 1A and 1B, the vanes 130 formed in the substrate 110 between the first side 112 and the second 114 define hexagonal-shaped filter regions 125 of a single filter membrane 120. For example, as illustrated in FIGS. 1A and 1B, the vanes 130 form a pattern of honeycomb-shaped cells 140. The filter membrane 120 disposed on the side 112 of the substrate 110 covers each honeycomb cell 140. Each honeycomb cell 140 (visible on side 114 of substrate 110 in FIG. 1A and visible through the filter membrane 120 in FIG. 1B) defines a hexagonal-shaped filter region 125 of the filter membrane 120. The pattern of cells formed by vanes 130 is not limited to the honeycomb pattern seen in FIG. 1A, however. For example, the vanes 130 can form a pattern of square cells (see FIG. 3A), rectangular cells (not illustrated), or cells of another shape.

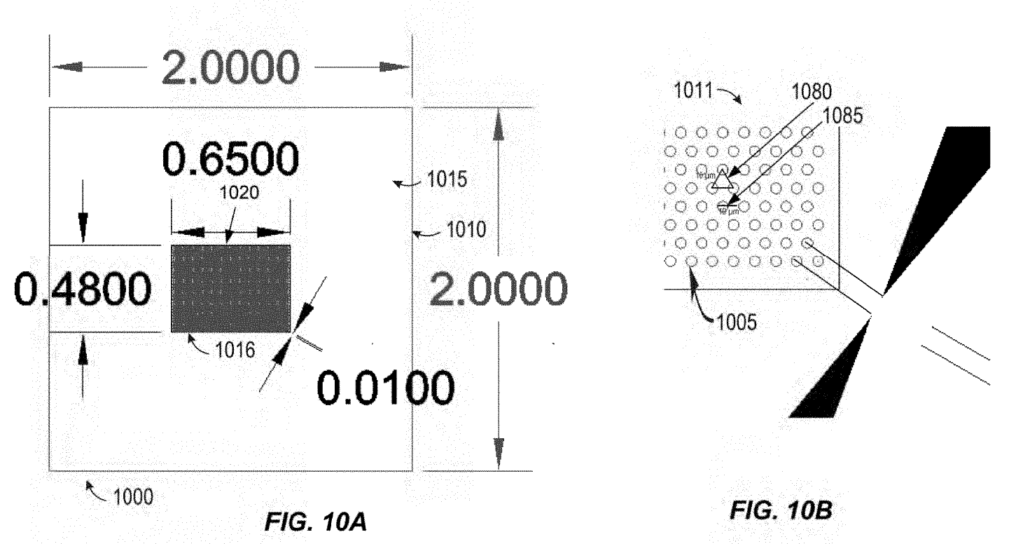

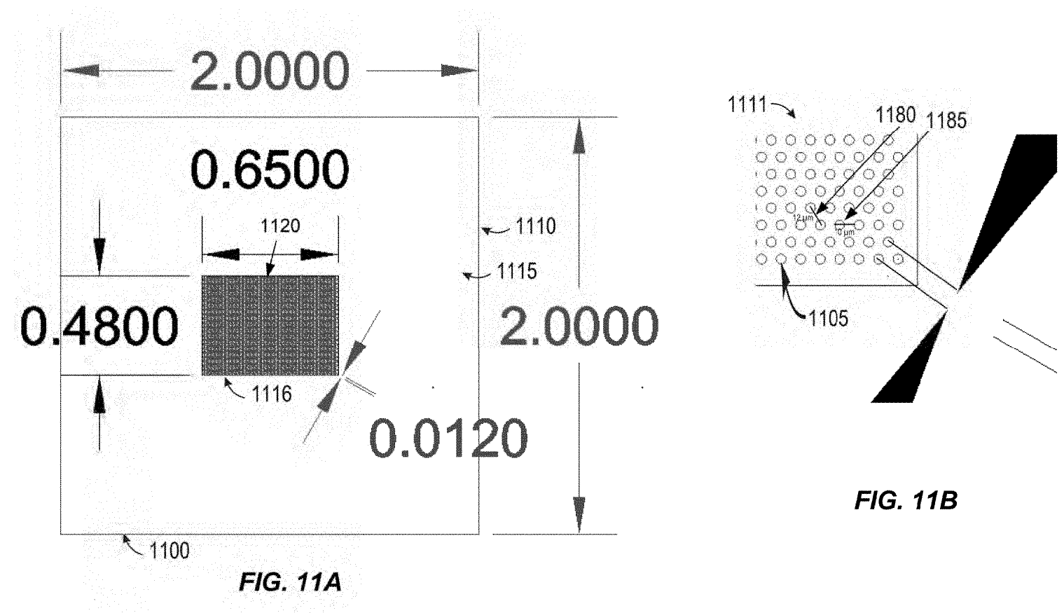

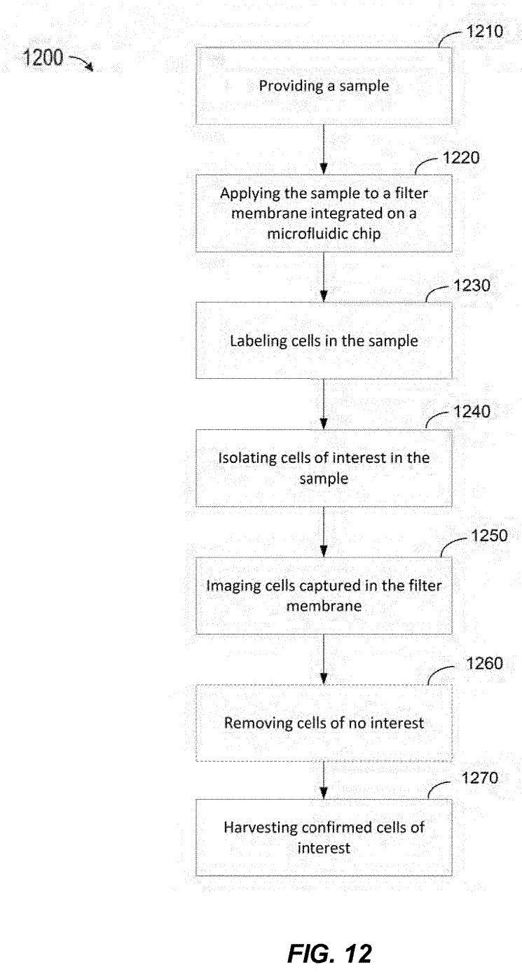

In some embodiments, vanes 130 are dimensioned and fabricated to provide support for the filter membrane 120. For example, the vanes 130 can support filter membrane 120 in a way that allows the filter membrane 120 to withstand a certain amount of pressure due to fluid flow. In the absence of vanes 130 in the substrate 110, the filter membrane 120 may sag, bend, or break due to the same amount of fluid being applied to a larger unsupported surface area of the filter membrane. In some aspects, vanes 130 advantageously provide further support and structural integrity for the filter membrane 120 than that provided by the sides 112, 114 of substrate 110, such that the middle of each filter membrane 120 does not sag, bend, or break due to pressures from fluid flow over and/or through the filter membrane 120. Further, vanes 130 forming honeycomb cells 140 can advantageously define a field of view (FOV) for imaging each hexagonal-shaped filter region 125 during an imaging cytometry process or other analysis as will be described below with reference to FIG. 12. As will be described in more detail below with reference to FIGS. 10A, 10B, 11A, and 11B, however, some embodiments of microfluidic chips described herein include substrates that do not have vanes or other supporting structures.

FIG. 1C illustrates a perspective view of a first side of another embodiment of a microfluidic device for capturing and positioning cells of interest according to the present disclosure. The microfluidic chip of FIG. 1C may be substantially similar to the microfluidic chip 100. However, the vanes supporting the filter membrane define rectangular-shaped filter regions (for example, the shaded-in filter region) of the filter membrane. The filter regions in this example microfluidic chip are arranged in a 5×5 grid-like pattern, as will be described in more detail below with reference to FIGS. 3, 4, 13, and 14.

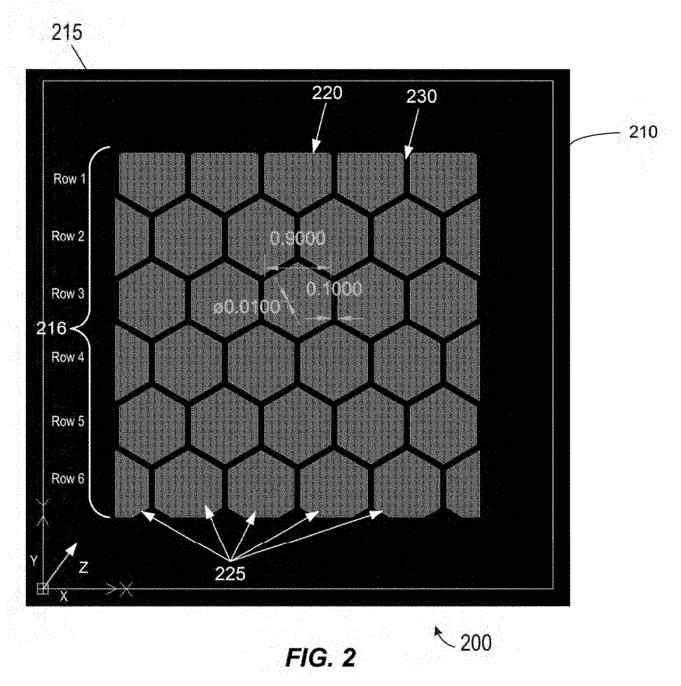

FIG. 2 illustrates an exemplary microfluidic chip 200 according to one embodiment. In this non-limiting example, the microfluidic chip 200 may be substantially similar to the microfluidic chip 100 having a support layer and a filter layer. In this case, the support layer includes a substrate 210 and the filter layer includes a filter membrane 220. The substrate 210 includes a frame-shaped exterior portion 215 and an interior portion 216 including vanes 230. The filter membrane 220 is positioned over and touching the vanes 230 in the interior portion 216. In this non-limiting example, the filter membrane is transparent such that the vanes 230 are visible through the filter membrane 220. The vanes 230 form a pattern of honeycomb-shaped cells (similar to honeycomb-shaped cells illustrated in microfluidic chip 100 of FIG. 1A). Other configurations are possible. In the example illustrated in FIG. 2, the vanes have a thickness of about 0.1 millimeter.

The vanes 230 define hexagonal-shaped filter regions 225 of filter membrane 220. It will be understood, however, that the microfluidic chip 200 can be designed to have filter regions 225 of any suitable shape (for example, hexagonal, square, rectangular, or any other shape). Advantageously, features of the number, size, and shape of the filter regions 225 can be selected to maximize capture of a particular cell of interest, based on the intended application for the microfluidic chip 200.

The filter membrane 220 includes a plurality of through holes arranged in a regularly-repeating pattern. The size, shape, and relative spacing of each through hole can be specifically selected based on the cell of interest the filter membrane is designed to capture and retain, such that a single cell of interest is captured and retained in each through hole. The through holes can have openings that are generally rectangular in shape, generally circular in shape, or any other suitable shape. In the non-limiting example illustrated in FIG. 2, the through holes have generally circular openings with diameters of about 10 microns.

One non-limiting advantage of filter membranes described herein is the ability to automatically create a monolayer of cells of interest as the sample flows through the filter membrane, which is not possible using a plating of the sample on a slide. Due to the specifically designed size, shape, and material properties of the through holes in the filter membranes, the filter membrane can be configured to prevent one potential cell of interest in the sample from obscuring, overlapping with, or lying on top of another potential cell of interest. As a result, imaging systems utilizing embodiments of the microfluidic chip described herein need not expend imaging resources, such as high resolution imaging resources, to determine where specific cell boundaries lie, to trace cell outlines to distinguish two closely-spaced cells from one another, or to ascertain if an object is actually two or more cells clumped together—activities that are typically required in conventional cell plating before a potential cell of interest is actually studied and confirmed to be a cell of interest.

In the non-limiting example depicted in FIG. 2, the substrate 210 includes an exterior portion 215 that is about 8 millimeters by about 8 millimeters measured along an x-axis and a y-axis of the microfluidic chip, and has a thickness of about 0.3 millimeter measured along a z-axis of the microfluidic chip. Other dimensions are possible. The interior portion 216 in this example is about 5 millimeters by about 5 millimeters measured along the x-axis and the y-axis of the microfluidic chip. The filter membrane 220 is positioned over and touching the substrate 210 in this example. The filter membrane 220 is about 5 μm measured along the z-axis of the microfluidic chip. Filter membranes having a different thickness are also suitable for use in the microfluidic chip 200.

In some cases, the hexagonal-shaped filter regions 225 defined by the vanes 230 can be referred to as the “active area” of the filter membrane 220. The areas of the filter membrane 220 that are disposed directly over and in contact with the vanes 230 are not considered “active areas” of the filter membrane 220 because the second openings of through holes in these areas may be blocked by the vanes 230, such that fluid flow through these through holes is degraded or entirely obstructed. In the non-limiting embodiment illustrated in FIG. 2, the hexagonal-shaped filter regions 225 are about 0.9 millimeter long measured along the x-axis of the microfluidic chip 200, separated by vanes 230 having a thickness of approximately 0.1 millimeter. The total area of one hexagonal-shaped filter region 225 is about 0.7 millimeter². Accordingly, each filter region 225 can represent an active area of about 0.7 millimeter². The interior portion 216 of the substrate 210 can define 6 rows of filter regions 225, labeled Row 1 through Row 6 in FIG. 2. There are approximately five filter regions 225 in Row 3 and 5. Additionally, the total area of filter regions 225 present in Row 2, for example, is equivalent to five filter regions 225.

### Example 1—Microfluidic Chip with Filter Membrane Having Rectangular Through Holes

FIG. 3A illustrates an exemplary microfluidic chip 300A according to one embodiment. In this non-limiting example, the microfluidic chip 300A includes a substrate 310 and a filter membrane 320. The substrate 310 includes a frame-shaped exterior portion 315 and an interior portion 316 including vanes 330. The substrate 310 includes an exterior portion 315 that is about 8 millimeters by about 8 millimeters measured along an x-axis and a y-axis of the microfluidic chip 300A. The substrate 310 has a thickness of about 0.4 millimeter measured along a z-axis of the microfluidic chip 300A. Other thicknesses are possible. The interior portion 316 in this example is about 5 millimeters by about 5 millimeters measured along the x-axis and the y-axis of the microfluidic chip 300A.

The filter membrane 320 is positioned over and touching the vanes 330 in the interior portion 316. The vanes 330 form a pattern of cube-shaped cells. Other configurations are possible. In the example illustrated in FIG. 3A, the vanes have a thickness of about 0.124 millimeter measured along the x-axis and the y-axis of the microfluidic chip 300A.

The vanes 330 define square-shaped filter regions 311 of the filter membrane 320. In this example implementation, the vanes 330 of microfluidic chip defines 25 filter regions 311 arranged in a 5×5 grid. The vanes 330 can define fewer than 25 filter regions in the filter membrane 320, such as 9 filter regions (as with a 9 filter regions arranged in 3×3 grid) or 16 filter regions (as with 16 filter regions arranged in a 4×4 grid). Some implementations include more than 25 filter regions, such as 64 or 100 filter regions. Other configurations are possible. Each filter region 311 of the filter membrane 320 defines an active region that is about 0.9 millimeter by about 0.9 millimeter measured along the x-axis and the y-axis of the microfluidic chip 300A.

As illustrated in the close-up view of one of the 25 filter regions, filter region 311A, the filter membrane 320 includes rectangular through holes, such as through hole 305, arranged in a regular, repeating pattern. However, it will be understood that any filter membrane described herein, not only that illustrated in FIG. 3A, may be included in microfluidic chip 300A depending on the cells sought to be captured, imaged, and analyzed in a particular application. The rectangular through holes in filter membrane 320 are about 5 μm high (measured along the y-axis of the microfluidic chip 300A) by about 10 μm long (measured along the x-axis of the microfluidic chip 300A). Through holes having other dimensions are possible as described in detail below. The rectangular through holes in this embodiment have rounded or chamfered corners. Rectangular through hole 305, for example, includes rounded corners each having a 1 μm radius.

In some example's, each through hole 305 of filter membrane 320 is spatially separated, or offset, from other through holes by a horizontal pitch of about 20 m (measured along the x-axis of the microfluidic chip) and a vertical pitch of about 10 m (measured in the y-axis of the microfluidic chip). The offset dimensions can be advantageously selected to maximize the number of through holes in filter membrane 320 without sacrificing structural integrity of the filter membrane 320, thus maximizing the number of cells that can be captured in the filter membrane 320. In some embodiments, the through hole dimensions are kept at 50% of the pitch dimensions. As described above, these through hole dimensions and spacing are examples and other configurations are possible based on the specific size and shape of the objects (such as cells or microbeads) of interest to be isolated in the microfluidic chip 300A. The size, shape, and relative spacing of each through hole 305 in microfluidic chip 300A can be specifically selected based on the object of interest (such as a cell) the filter membrane 320 is designed to capture, such that a single object of interest is captured in each through hole 305.

In one example, a rectangular through hole 305 may be dimensioned to capture a single RBC in the through hole based on the general disk-like shape of RBCs. In another example, a rectangular through hole 305 may be dimensioned to allow mature disk-shaped RBCs (such as maternal RBCs) to pass through the through hole 305, while a single fetal nucleated RBC is captured and retained in a single through hole 305 based on the spherical shape and slightly larger size of fetal nucleated RBCs. Thus, the characteristics and dimensions of each through hole may be specifically selected based on the shape and size of the cells of interest. Further, the density of through holes on a single filter membrane, and the relative position or arrangement of the through holes relative to each other, can be selected to optimize the number of cells of interest that are retained or “captured” in the filter membrane. Advantageously, the orientation of through holes in filter membranes described herein can be rotated, flipped, or shifted such as to maximize the number of through holes exposed to cells in the sample, thereby maximizing the number of cells of interest captured by the filter membrane.

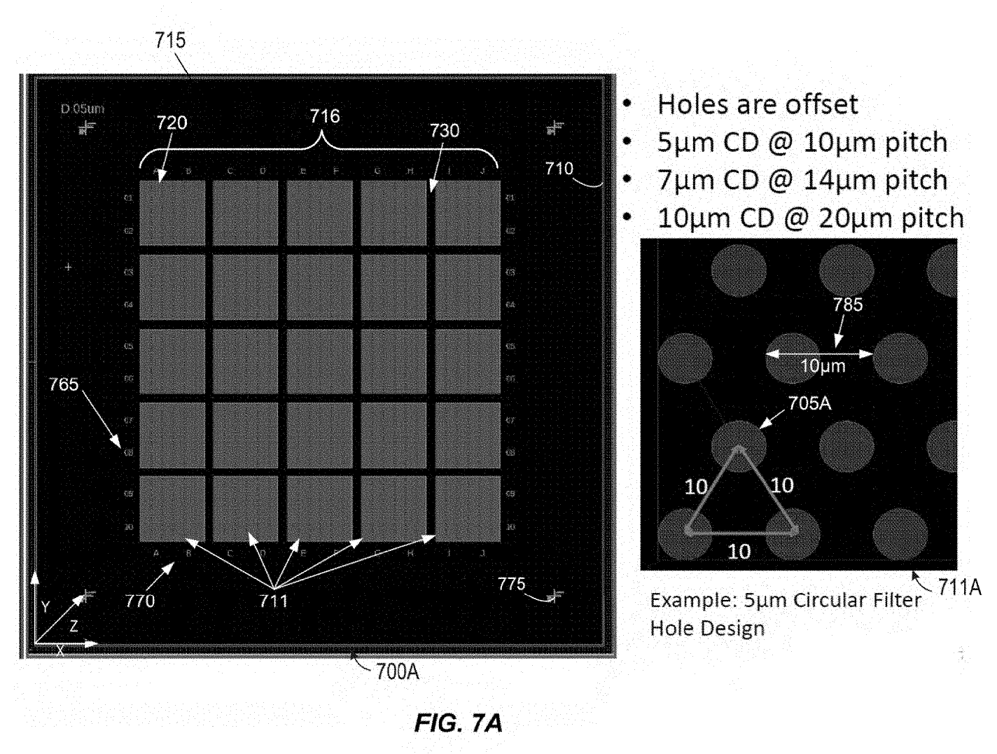

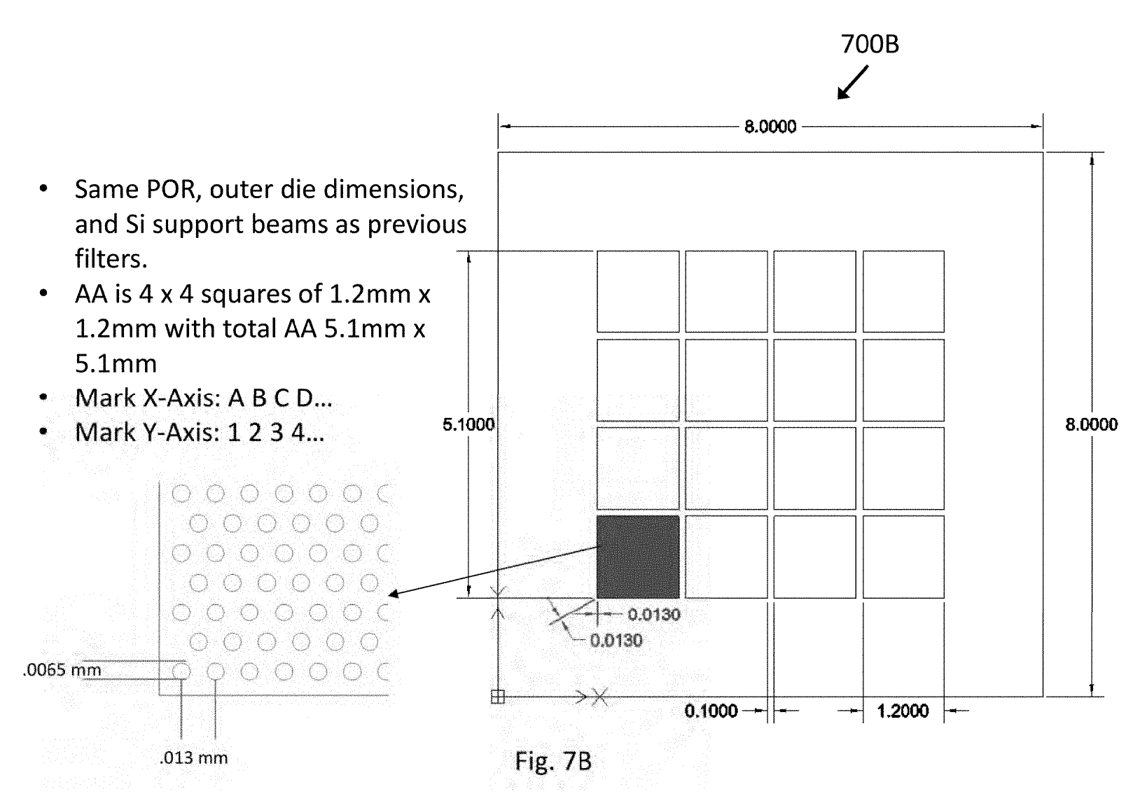

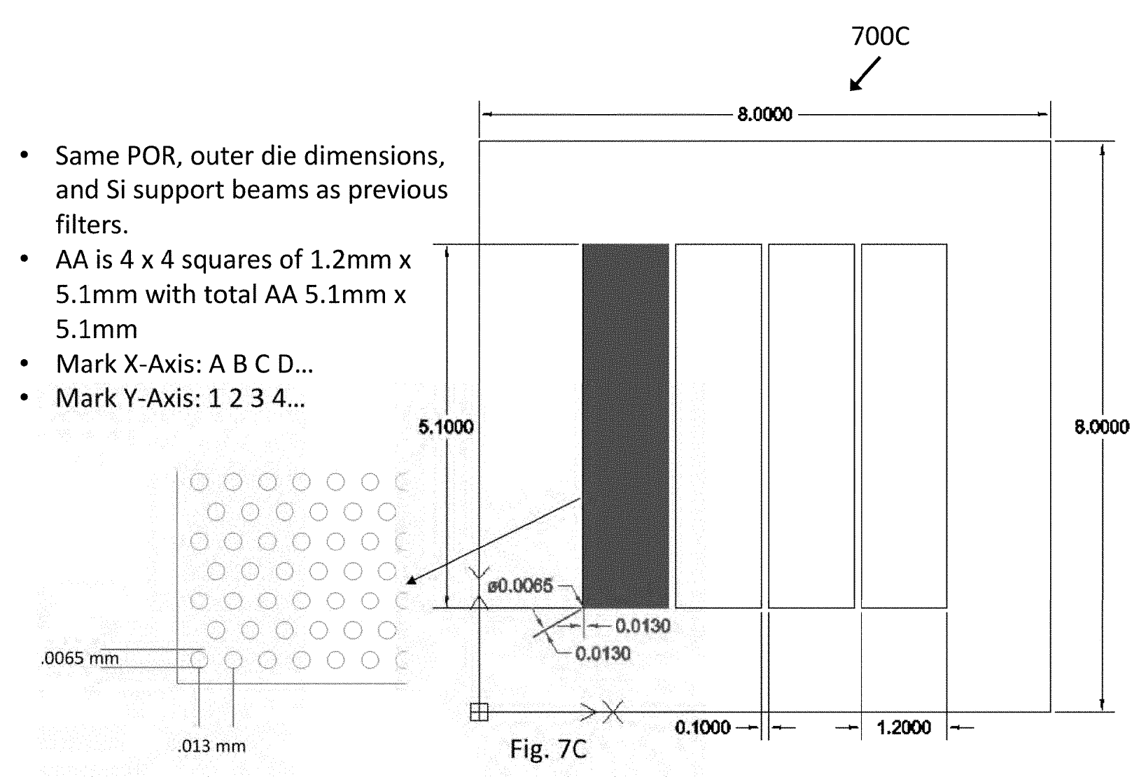

In other embodiments, the through hole 305 may have an opening that is generally circular (for example, as described in greater detail below with reference to FIGS. 7 through 9C). The circular through holes may be specifically designed and configured to capture any desired cell, microbead, or other object based on known characteristics (such as, but not limited to, the size and morphology) of the sought after cell, microbead, or other object. By changing the shape and size of the through holes, multiple filter membranes can be designed and manufactured for the isolation of specifically sought after cells or objects. In one non-limiting example, a filter membrane is designed to include circular holes that are shaped and sized to capture specifically-identified bacterial cells of interest. In one non-limiting example, circular through holes have a diameter of about 10 μm. Other dimensions are possible. For example, through holes 305 can have a diameter of about 5 μm or a diameter of about 7 μm. For example, through holes 305 can have a diameter of approximately 6.5 μm. In various embodiments, the through holes 305 can be circular and can have a diameter in the range of about 4 μm to about 10 μm.

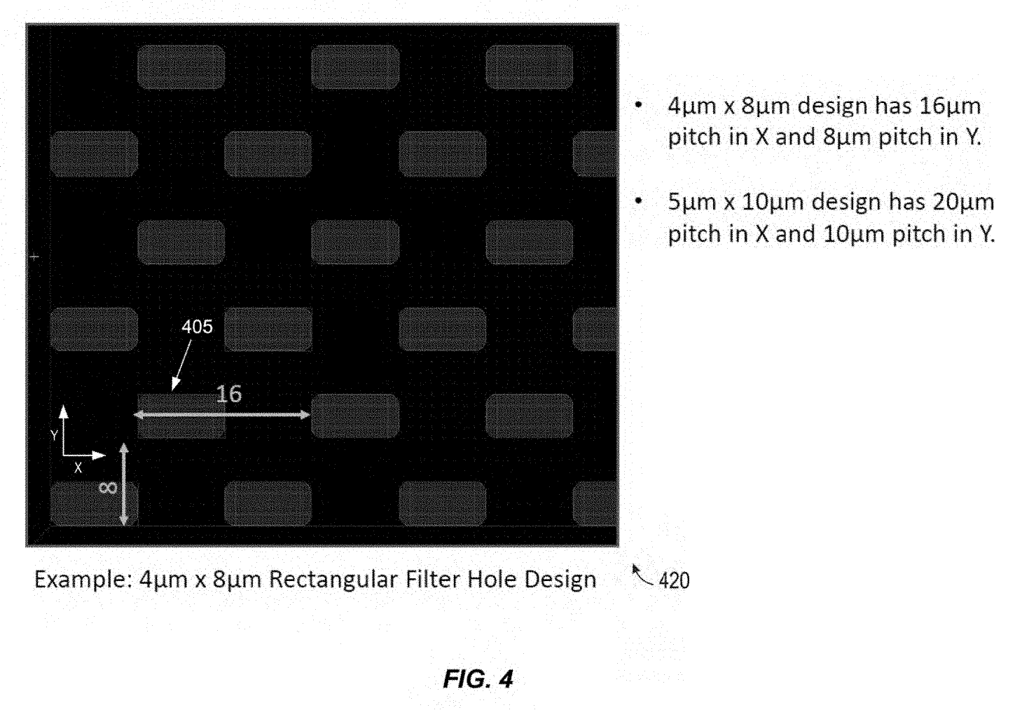

FIG. 4 illustrates another exemplary filter membrane having a regular, repeating pattern of rectangular through holes. Specifically, FIG. 4 illustrates a close-up view of one portion of a filter membrane 420 having a plurality of through holes 405. A microfluidic chip according to embodiments described herein may include a plurality of filter membranes 420, or a single filter membrane 420 (as described above with reference to FIG. 3A). In the embodiment illustrated in FIG. 4, the filter membrane 420 includes rectangular-shaped through holes 405 that are about 4 μm high (measured along the y-axis of the microfluidic chip) by 8 μm long (measured along the x-axis of the microfluidic chip). Other dimensions are possible.

Each through hole 405 of filter membrane 420 is offset from other through holes by a horizontal pitch of about 16 μm (measured along the x-axis of the filter membrane 420) and a vertical pitch of about 8 μm (measured along the y-axis of the filter membrane 420). The offset dimensions can be selected to maximize the number of through holes in filter membrane 420 without sacrificing structural integrity of filter membrane 420, thus maximizing the number of cells that can be captured in the filter membrane 420.

The above-described through hole dimensions and spacing are examples and other configurations are possible based on the specific size and shape of the objects (such as cells or microbeads) of interest that the filter membrane 420 is intended to capture. For example, the filter membrane 420 can include through holes that are about 5 μm high (measured along the y-axis of the microfluidic chip) by about 10 μm long (measured along the x-axis of the microfluidic chip). The through holes can be offset from each other by a horizontal pitch of about 20 μm (measured along the x-axis of the filter membrane 420) and a vertical pitch of about 10 μm (measured along the y-axis of the filter membrane 420).

The rectangular through holes 405 in this embodiment advantageously include rounded or chamfered corners. Rectangular through holes including rounded corners enhance fluid flow through the filter membrane 420. Without being bound to any particular theory, it is believed that he rounded or chamfered corners remove dead spots in the fluid flow through the through hole 405 that would ordinarily occur if the corners of the through hole included sharp angular edges. These sharp angular corners may cause the accumulation of fluid and/or cells within or around the corner. In this way, embodiments of through holes described herein can advantageously permit smooth flow of fluid through the filter.

FIG. 3B illustrates another exemplary microfluidic chip 300B according to one embodiment. The microfluidic chip 300B illustrated in FIG. 3B has rectangular-shaped through holes, similar in cross-sectional shape to through holes included in microfluidic chip 300A depicted in FIG. 3A. The through holes in the microfluidic chip 300B are about 0.008 millimeters long measured along a y-axis of the microfluidic chip and are about 0.004 millimeters long measured along an x-axis of the microfluidic chip. The microfluidic chip 300B is about 8 millimeters by about 8 millimeters measured along the x-axis and y-axis of the microfluidic chip, and has an active area that is about 5.1 millimeters by about 5.1 millimeters. The plurality of filters are arranged in a 4×4 grid-like pattern. Each filter is about 1.2 millimeters by about 1.2 millimeters measured along the x-axis and the y-axis of the microfluidic chip. The filters are separated by vanes that are about 0.1 millimeters wide measured along the x-axis of the microfluidic chip 300B. Other configurations are possible.

FIG. 3C illustrates another exemplary microfluidic chip according to one embodiment. The microfluidic chip 300C in FIG. 3C has rectangular-shaped through holes, similar in cross-sectional shape to through holes included in microfluidic chips 300A and 300B. The microfluidic chip 300C is about 8 millimeters by about 8 millimeters measured along the x-axis and y-axis of the microfluidic chip 300C. The filters included in microfluidic chip 300C are manufactured with different dimensions than the filters in the microfluidic chips 300A and 300B. The plurality of filters in microfluidic chip 300C are arranged in a 4×1 grid-like pattern and are about 1.2 millimeters measured along an x-axis of the microfluidic chip by about 5.1 millimeters measured along a y-axis of the microfluidic chip. Other configurations are possible.

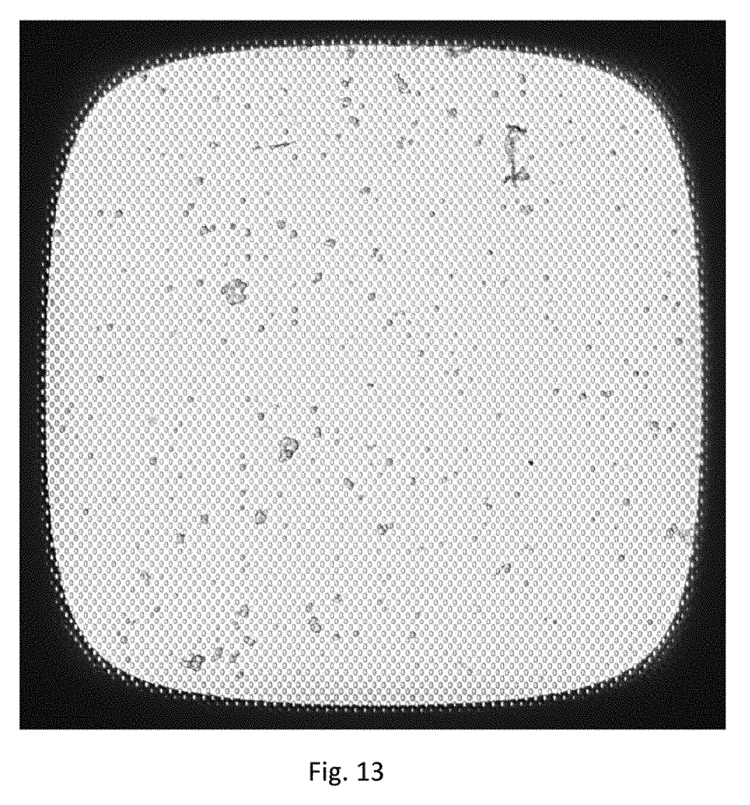

FIG. 13 illustrates an example image taken of one filter membrane of a microfluidic chip used to capture cells of interest in through holes having a rectangular shape. FIG. 13 depicts a filter membrane that may be substantially similar one of the filters of microfluidic chip 300A. FIG. 13 is an actual image of a filter taken by a microscope platform, where cells of interest in a sample or portions of the sample are captured and retained in precisely-defined and identifiable through holes on the filter.

### Example 2—Microfluidic Chip with Filter Membrane Having Oval Through Holes

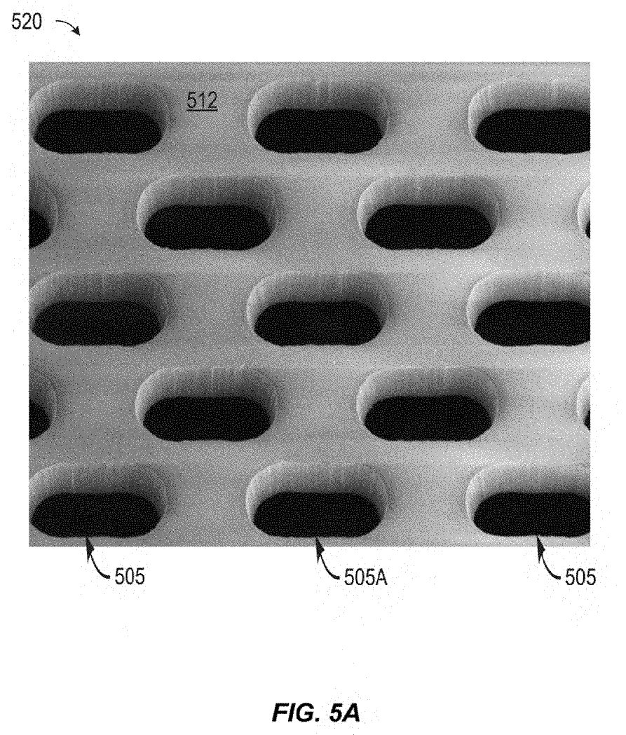

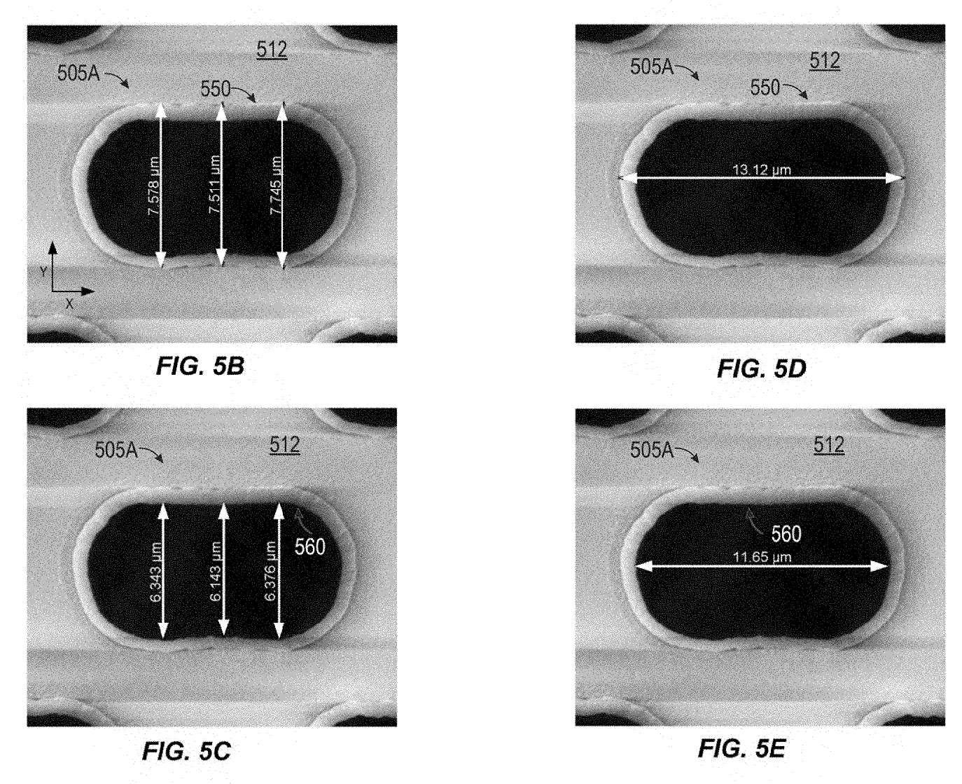

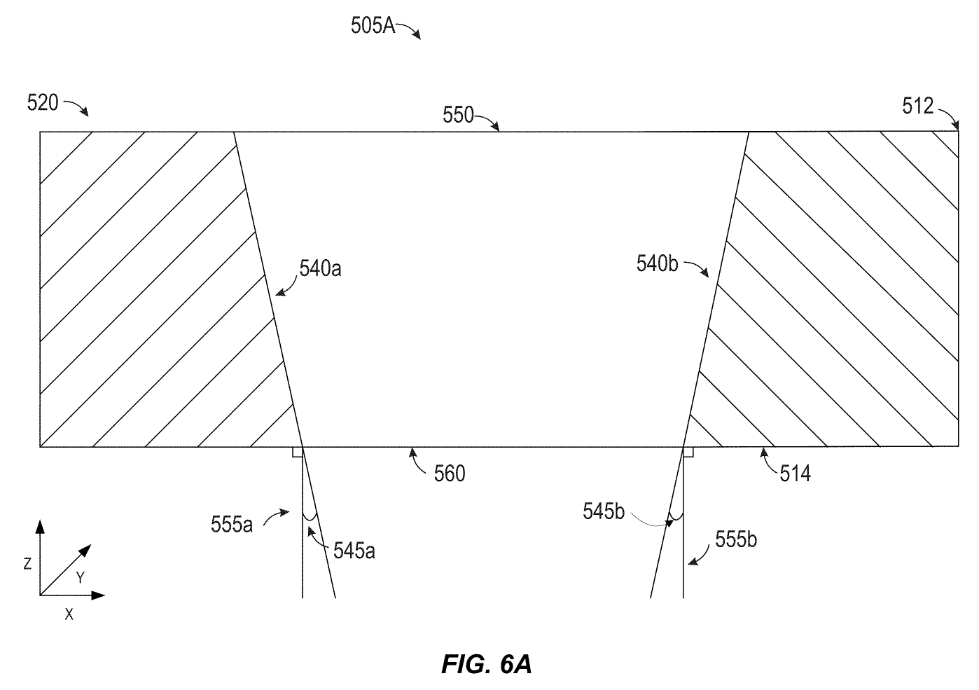

FIG. 5A is an image taken of a portion of a filter membrane 520 having a regular, repeating pattern of through holes 505 according to the present disclosure. FIGS. 5B, 5C, 5D, and 5E are close-up images of a single through hole 505A of the filter membrane 520 of FIG. 5A. A microfluidic chip comprising filter membrane 520, or multiple filter membranes 520, may be substantially similar to the microfluidic chips described herein. For example, although not depicted in FIG. 5A because only a portion of filter membrane 520 has been imaged, the filter membrane 520 can include an active area that is about 5 millimeters by about 5 millimeters. Additionally, embodiments of the filter membrane 520 can be supported by a substrate having dimensions described herein, such as a substrate having a frame-shaped exterior portion that is about 8 millimeters by about 8 millimeters.

A plurality of through holes 505 having generally oval-shaped openings are arranged in a regular, repeating pattern in the filter membrane 520. The through holes 505 are configured to capture and simultaneously position objects of interest (such as cells of interest) in precisely-defined, clearly-distinguishable locations on the filter membrane 520 (each location corresponding to a single through hole 505). In this non-limiting example, the filter membrane 520 was designed and manufactured to include through holes 505 that are generally about 5 μm high (measured along the y-axis of the filter membrane 520) by about 10 μm long (measured along the x-axis of the filter membrane 520). As will be described in detail below, however, the actual dimensions of a single through hole, such as through hole 505A depicted in FIGS. 5B through 5E, may vary slightly from this target 5 μm by 10 μm dimension. The desired dimension (such as 5 μm in the y-direction, or 10 μm in the x-direction) may be referred to as a “target dimension,” of the through hole, indicative of a minimum allowable dimension of a finished through hole. Thus, the actual, manufactured dimensions of through holes 505 may not be less than the target dimensions of 5 μm by 10 μm. Otherwise, the through holes 505 may unintentionally capture objects in a fluid sample that are not objects of interest (for example, a mature maternal RBC may be captured in a through hole 505 that has finished dimensions less than about 5 μm by about 10 μm, even though such a RBC is not a cell of interest).

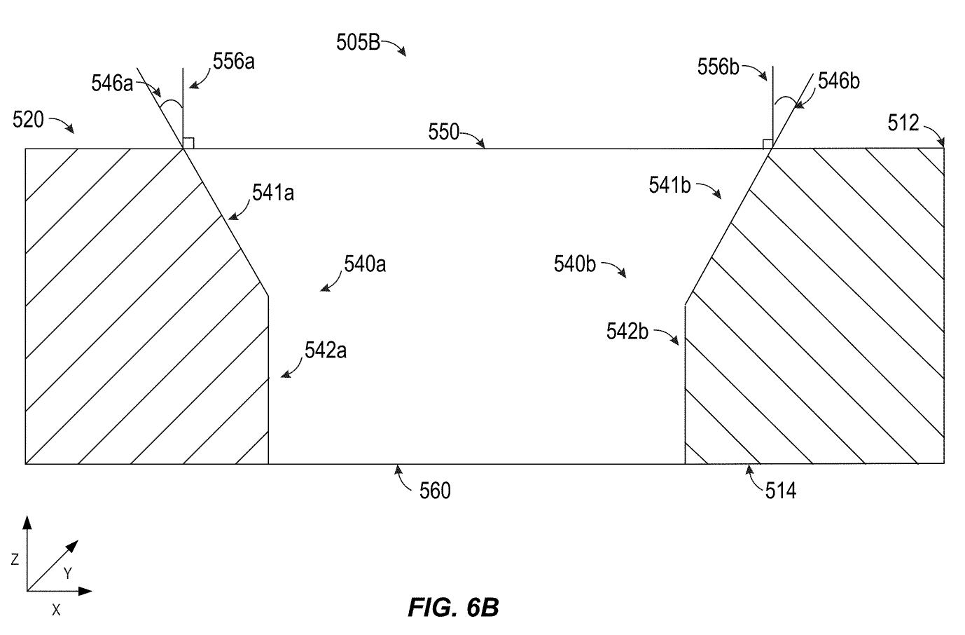

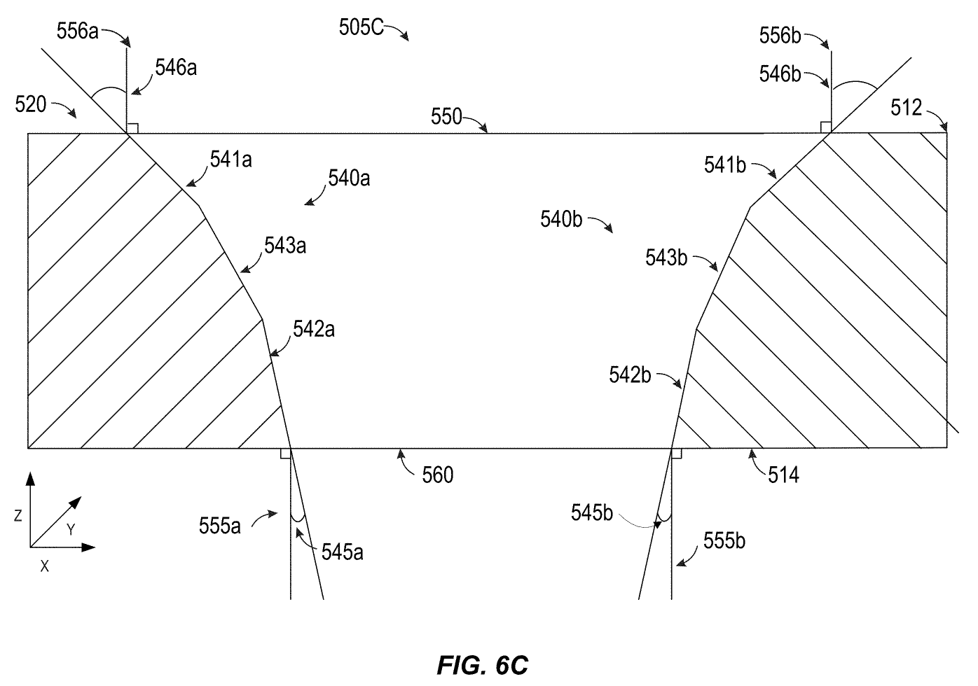

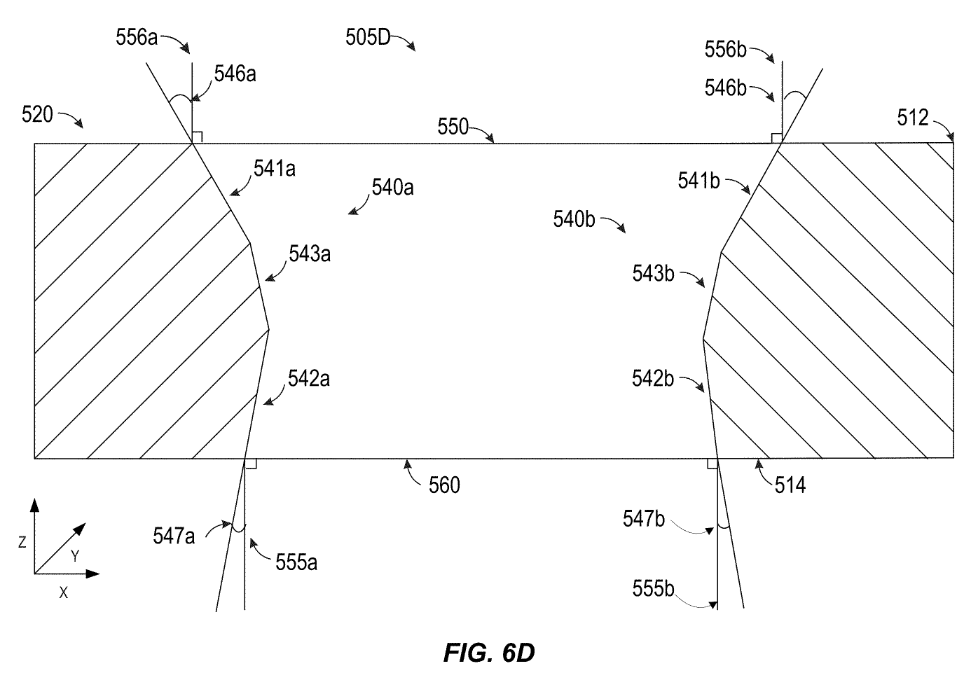

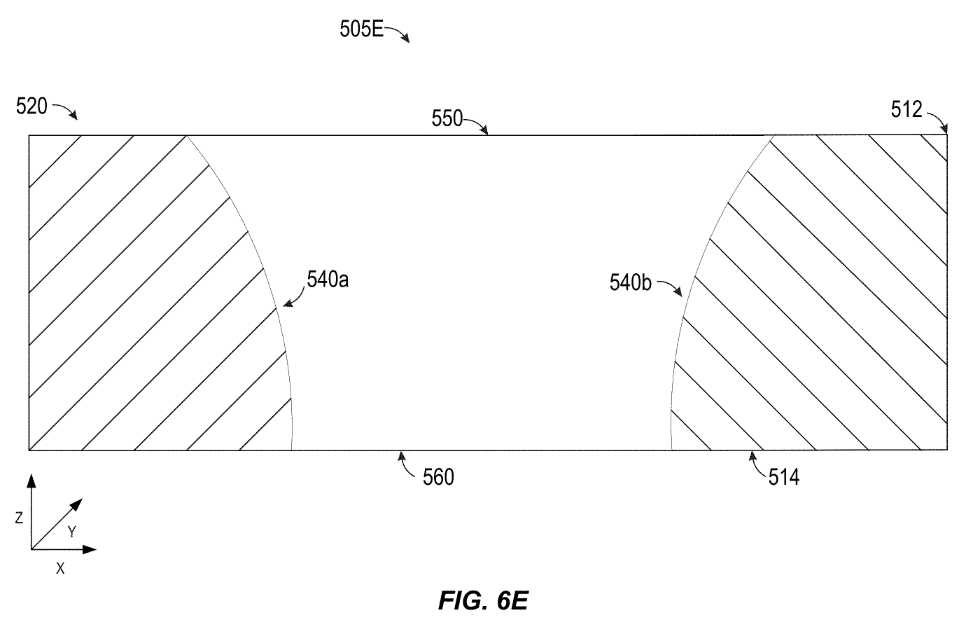

The sidewalls of the through holes 505 extending through the interior of filter membrane 520 are advantageously angled or tapered, as illustrated in FIG. 6A. FIG. 6A depicts a cross-sectional view of the through hole 505A in filter membrane 520. FIG. 6A is a schematic representation and is not drawn to scale. The through hole 505A includes sidewalls 540a and 540b extending between a first side 512 and a second side 514 of the filter membrane 520, thereby allowing objects to translocate through the filter membrane 520. As illustrated in FIG. 6A, the sidewall 540a is tapered at an angle 545a relative to line 555a that is perpendicular to the second side 514 of filter membrane 520. The sidewall 540 is also tapered at an angle 545b relative to line 555b that perpendicular to the second side 514 of filter membrane 520. In this non-limiting example, the sidewalls 540a, 540b are tapered at an angle of about 12° relative to lines 555a, 555b, respectively. The tapered sidewalls 540a and 540b may be configured such that a first opening 550 of the through hole 505A located on the first side 512 of the filter membrane 520 is larger than a second opening 560 of the through hole 505A located on the second side 514 of the filter membrane 520. Through holes having tapered sidewalls such as tapered sidewalls 540a and 540b can capture more cells of interest in a sample flowing through the filter membrane 520, and in some cases can retain captured cells of interest more securely (while additional fluid samples are passed through the filter, for example). Without being bound to any particular theory, it is believed that first openings (such as first opening 550) with larger dimensions permit cells of interest to more freely and consistently enter the through hole 505A while second openings (such as second opening 560) with smaller dimensions inhibit the cell of interest from passing entirely through the second opening 560 and out the second side 514 of the filter membrane 520, thereby facilitating improved capture of the cell in the through hole 505A.

In one non-limiting embodiment, the features of an angled sidewall of a through hole serve dual functions: one being a physical hydrodynamic trap preventing captured cells or beads of further lateral or directional movement, and the other being a filtering or isolating membrane. If the through holes do not include tapered side walls, the through holes may only serve to prevent certain cells from flowing through the filtering membrane, but would not serve as a hydrodynamic trap, or capturing grid, for cells or beads of targeted size, thus retaining and immobilizing them within, or partially within, the through holes. In one non-limiting example of a circular through hole, the thickness of the membrane and the angle of the through hole side walls determine the capturing and immobilizing characteristics of the filter membrane, and the smallest diameter, at the bottom of the through hole, determines its filtering or isolating properties. Similar effects can be achieved in non-circular through holes by independently selecting the angle of the tapered side wall and the smallest dimension (measured along the x-axis and the y-axis) of the through hole.

In some embodiments, the angles 545a and 545b are substantially equal, while in other embodiments the sidewalls 540a and 540b may be tapered at different angles due to design specifications or manufacturing variances. In some embodiments, the angles 545a and 545b can be between approximately 0 degrees and approximately 20 degrees. In the non-limiting embodiment illustrated in FIG. 6A, for example, the angles 545a and 545b are approximately 12 degrees. In another embodiment, the angles 545a and 545b can be between approximately 5 degrees and approximately 10 degrees. The angles 545a and 545b may be selected depending on the particular application and cells of interest to be captured in the filter membrane. The angles 545a and 545b and tapered sidewalls 540a and 540b do not result in either the first opening 550 nor the second opening 560 having a dimension that is less than the target dimension, however.

Other variations of tapered sidewalls in accordance with the present description will now be described with reference to FIGS. 6B, 6C, and 6D. In some embodiments, the sidewalls of through hole 505 extending through the interior of filter membrane 520 may be angled or tapered with one or more angles that may be varied relative to the line that is generally perpendicular to the first and/or second side 512 and 514 of the filter membrane 520, as illustrated in FIGS. 6B through 6D. FIGS. 6B through 6D depict cross-section views of various embodiments of through holes 505B through 505D in a filter membrane 520. FIGS. 6B through 6D are schematic representations that may be similar to the through hole 505A of FIG. 6A and are not drawn to scale. However, FIGS. 6A through 6D illustrate through holes 505A through 505D having two or more regions (e.g., FIG. 6B shows two regions 541a, 541b and 542 a, 542b and FIGS. 6C and 6D show three regions 541a, 541b; 542a, 542b; and 543a, 543b), where each region may be tapered at a different angle (e.g., 545a, 545b; 546a, 546b; and 547a, 547b) relative to the line (e.g., 555a, 555b, 556a, and/or 556b) generally perpendicular to the first and/or second side 512 and 514 of the filter membrane 520. In some embodiments, there may be 1, 2, 3, 4, or more regions along the tapered sidewalls 540a and 540b, each being tapered at a different angle relative to the line generally perpendicular to the first and/or second side of the filter membrane.

FIG. 6E illustrates an embodiment of through hole 505E including sidewalls 540a and 540b that are curved with a radius of curvature. FIG. 6E is a schematic representation that may be similar to the through hole 505A of FIG. 6A and is not drawn to scale. In some embodiments, the radius of curvature may be constant along sidewalls 540a and 540b between first and second surface 512 and 514 of filter membrane 520. In other embodiments, the radius of curvature may vary along sidewalls 540a and 540b. For example, the various embodiments of FIGS. 6A through 6E may be combined, such that sidewalls 540a and 540b have multiple regions, where each region may be curved at some radius (each radius may be different or the same) and/or be tapered at various angles relative to the line generally perpendicular to the first and/or second side of the filter membrane.

Turning now to FIGS. 5B, 5C, 5D, and 5E, close-up images of the through hole 505A of the filter membrane 520 of FIG. 5A are provided. Through hole 505A having tapered sidewalls 540a, 540b is representative of the through holes 505 in the filter membrane 520 of FIG. 5A. FIGS. 5B and 5D are images of the first opening 550 of the through hole 505A taken from the first side 512 of the filter membrane 520. FIGS. 5C and 5E are also images taken from the first side 512 of the filter membrane 520, but are intended to demonstrate the actual dimensions of second opening 560 that is located on the second side 514 of the filter membrane 520.

As discussed above, through hole 505A has a target height dimension of 5 μm measured in the y-direction of the filter membrane 520 (in other words, the filter membrane 520 is designed to include through holes 505 with first openings 550 and second openings 560 that are no less than 5 μm high in the y-direction). As illustrated in the image provided in FIG. 5B, however, the actual height of the opening 550 on the first side 512 of the through hole 505 measured in the y-direction varies along the x-axis, and the y-direction height at each of three measurements is greater than target dimension of 5 μm. For example, the measured height of the opening 550 in the y-direction is 7.578 μm, 7.511 μm, and 7.745 μm at three different points along the x-axis. Additionally, as illustrated in the image provided in FIG. 5C, the actual height of the opening 560 on the second side 514 measured in the y-direction also varies along the x-axis, and the y-direction height at each of three measurements is greater than the target dimension of 5 μm. For example, the measured height of the opening 560 in the y-direction is 6.343 μm, 6.143 μm, and 6.376 μm at three different points along the x-axis. Further, the inclusion of tapered sidewalls of 505A described above with reference to FIG. 6A are evident from the measurements illustrated in FIGS. 5B and 5C, as the average height of opening 550 on the first side 512 of through hole 505A is larger than the average height of opening 560 on the second side 514 of through hole 505A.

As illustrated in the image of opening 550 provided in FIG. 5D, the actual length of first opening 550 on the first side 512 of through hole 505A in the x-direction is approximately 13.12 μm. As illustrated in the image of FIG. 5E, the actual length of opening 560 on the second side 514 of through hole 505A is approximately 11.65 μm. Accordingly, the lengths of openings 550 and 560 measured in the x-direction are both greater than the target length dimension of 10 μm. Additionally, the inclusion of tapered sidewalls of 505A described above with reference to FIG. 6A are evident from the measurements illustrated in FIGS. 5D and 5E, as the length of opening 550 on the first side 512 of through hole 505A is larger than the length of opening 560 on the second side 514 of through hole 505A.

### Example 3—Microfluidic Chip with Filter Membrane Having Circular Through Holes

FIG. 7A illustrates an exemplary microfluidic chip 700A according to one embodiment. In this non-limiting example, the microfluidic chip 700A includes a substrate a 710 and a filter membrane 720. The substrate 710 includes an exterior portion 715 and an interior portion 716 that is substantially similar to the exterior portion 315 and interior portion 316 described with reference to FIG. 3A. Specifically, the substrate 710 includes a frame-shaped exterior portion 715 and an interior portion 716 including vanes 730. As will be described in detail below, however, the filter membrane 720 in microfluidic chip 700A includes through holes 705 that differ from the through holes 305 in filter membrane 320 in microfluidic chip 300A.

The frame-shaped exterior portion 715 in this embodiment includes position identifiers “01” through “10” along a vertical edge 765 of the interior portion 716, and position identifiers “A” through “J” along a horizontal edge 770 of the interior portion 716. The position identifiers can be labels that identify the position of each filter region 711 relative to the other filter regions 711. By referencing the identifiers along the vertical edge 765 and the horizontal edge 770, the precise location of each filter region 711 in the filter membrane 720 can be identified. Such information can be advantageously used during a process, described in detail below with reference to FIG. 12, when objects captured in the filter membrane 720 are imaged and analyzed to determine if they are objects of interest, and when confirmed objects of interest are harvested from the filter membrane.

In this embodiment, the precise location of each through hole 705 is known with reference to a specific x and y coordinate relative to one or more markers 775 on the surface of substrate 710. The markers 775 in this embodiment are located in the exterior portion 715 near the four corners of the substrate 710. Other locations are possible. The known, precisely-defined x, y location of each through hole 705 in the filter membrane 720 allows each through hole 705 to be identified, located, and re-located on the filter membrane 720 during multiple different steps of an imaging and analysis process described below with reference to FIG. 12. For example, the precise x, y location of a through hole 705A in which an object is captured may be initially recorded during an initial analysis step. This initial analysis step may include taking low resolution images of each filter region 725 to ascertain which through holes 705 have captured objects. The precise x, y location of this through hole 705A may then be used in a later step, for example, when an imaging platform takes high resolution images of only the through holes 705 that have been identified as likely to contain an object of interest. After the object captured in through hole 705A has been confirmed to be an object of interest, the precise x, y location of this through hole 705A may again be used to direct a harvesting platform, such as a needle, to the exact location of through hole 705 for removal of the confirmed object of interest from the through hole 705A.

It will be understood that the position identifiers and markers of microfluidic chip 700A in FIG. 7A can be applied to all microfluidic chips described herein. Additionally, other mechanisms to identify, label, and locate a particular filter region 725 and a particular through hole 705 are possible, and are not limited to the position identifiers 765, 770 and markers 775 described above.

The microfluidic chip 700A also includes a filter membrane 720 positioned over and touching the vanes 730 in the interior portion 716 of the substrate 710. In this non-limiting embodiment, the vanes 730 form a pattern of cube-shaped cells. Other configurations are possible (such as, but not limited to, the honeycomb-shaped cells 140 described above with reference to FIG. 1A). The vanes 730 define square-shaped filter regions 711 of the filter membrane 720. In this example implementation, the vanes 730 of the substrate 710 define 25 filter regions 711 arranged in a 5×5 grid. The vanes 730 of microfluidic chip 700A can define fewer or more filter regions, depending on the particular application for the microfluidic chip 700A. Each filter region 711 of filter membrane 720 defines an active region that is substantially similar to the active region defined by filter regions 311 described in reference to FIG. 3A. In this non-limiting aspect, each filter region 711 is about 0.9 millimeters by about 0.9 millimeters measured along the x-axis and the y-axis of the microfluidic chip 700A, and the total active region of filter membrane 720 is about 20.25 millimeters². Other configurations are possible.

As illustrated in the close-up view of one of the 25 filter regions, filter region 711A, the filter membrane 720 includes circular through holes, such as through hole 705A, arranged in a regular, repeating pattern. However, it will be understood that any filter membrane described herein, not only that illustrated in FIG. 7A, may be included in microfluidic chip 700A depending on the cells sought to be captured, imaged, and analyzed in a particular application. The circular through holes in filter membrane 720 are generally 5 μm in diameter. Through holes having other dimensions are possible, for example about 7 μm in diameter or about 10 μm in diameter. In one embodiment, the filter membrane 720 includes through holes that are about 6.5 μm in diameter.

Each through hole 705 of filter membrane 720 is spatially separated, or offset, from other through holes by a center-to-center pitch of about 10 μm. For example, each grouping of three through holes 705 form an equilateral triangle with three sides of about 10 μm length connecting the centers of each through hole 705. The lengths of the sides of this equilateral triangle may be referred to as the “pitch” of the through holes 705. Each through hole 705 can also be separated or “offset” from adjacent through holes 705 in the same row by an offset distance 785 of about 10 Atm. The pitch and offset of the through holes 705 can be advantageously selected to maximize the number of through holes in filter membrane 720 without sacrificing structural integrity of the filter membrane 720, thus maximizing the number of cells that can be captured in the filter membrane 720. As described above, the above-described through hole dimensions, offset spacing, and pitch are examples and other configurations are possible based on the specific size and shape of the objects (such as cells or microbeads) of interest to be isolated in the microfluidic chip 700A. In one example, through holes are spaced relative to each other with a pitch and an offset distance that are roughly double the target dimension of the through holes, where the target dimension for a circular through hole is the smallest diameter of the through hole. Thus, circular through holes having diameters equal to about 7 μm may be separated by a pitch of about 14 μm and an offset distance of 14 μm, whereas circular through holes having a diameter equal to about 10 μm may be separated by a pitch of about 20 μm and an offset distance of 20 μm. Other configurations are possible.

The size, shape, and relative spacing of each through hole 705 in microfluidic chip 700A can be specifically selected based on the object of interest (such as a cell) the filter membrane 720 is designed to capture, such that a single object of interest is captured in each through hole 705. In one exemplary filter 700A according to another embodiment, a filter membrane 720 comprising circular through holes may be specifically designed and configured to capture any desired cell, microbead, or other object based on known characteristics (such as, but not limited to, the size and morphology) of the sought after cell, microbead, or other object. By changing the shape and size of the through holes, multiple filters can be designed and manufactured for the isolation of specifically sought after cells or objects. In one non-limiting example, a filter membrane 720 included in an integrated microfluidic chip is designed to include through holes with circular openings that are shaped and sized to capture specifically identified bacterial cells of interest. Thus, the characteristics and dimensions of each through hole may be specifically selected based on the shape and size of the cells or objects of interest. Further, the density of through holes on a single filter membrane, and the relative position or arrangement of the through holes relative to each other, can be selected to optimize the number of cells of interest that are retained or “captured” in the filter membrane.

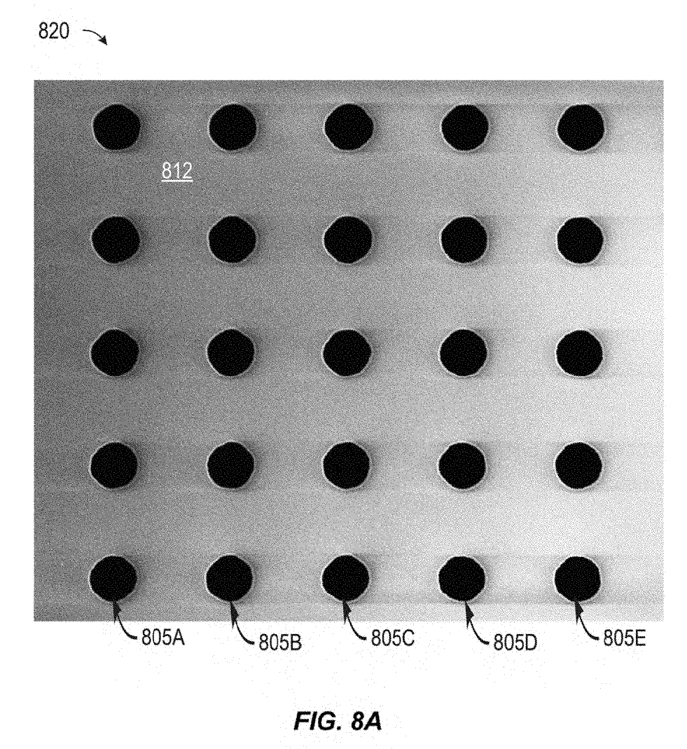

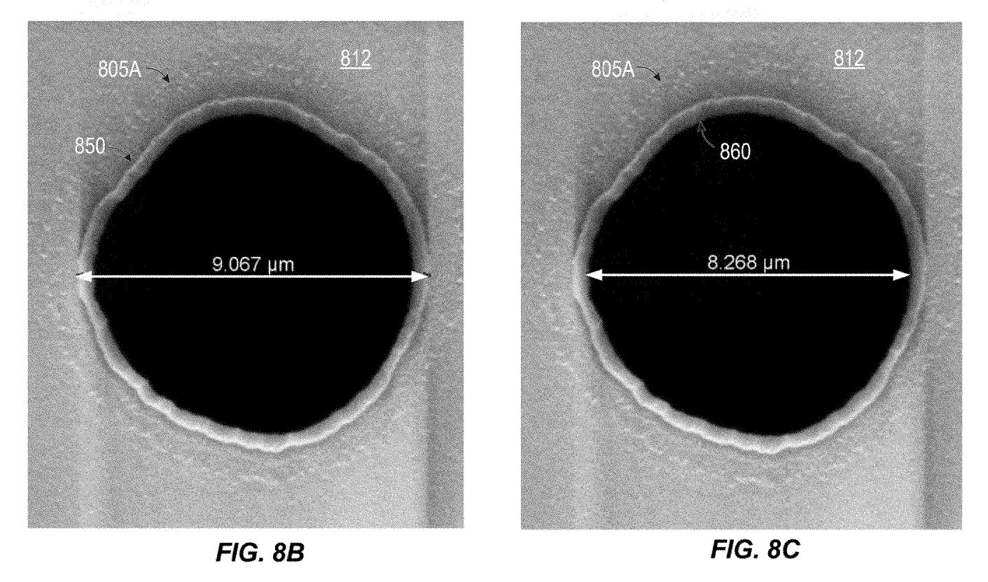

FIG. 8A is an image taken of a portion of a filter membrane 820 having a regular, repeating pattern of through holes 805 according to the present disclosure. FIGS. 8B and 8C are close-up images of a single through hole 805A of the filter membrane 820 of FIG. 8A. A microfluidic chip comprising filter membrane 820, or multiple filter membranes 820, may be substantially similar to the microfluidic chips described herein. For example, although not depicted in FIG. 8A because only a portion of filter membrane 820 has been imaged, the filter membrane 820 can include an active area that is about 5 millimeters by about 5 millimeters measured along an x-axis and a y-axis of the filter membrane 820. Additionally, embodiments of the filter membrane 820 can be supported by a substrate having dimensions described herein, such as a substrate having a frame-shaped exterior portion that is about 8 millimeters by about 8 millimeters.

A plurality of through holes 805 having generally circular openings are arranged in a regular, repeating pattern in the filter membrane 820. The through holes 805 are configured to capture and simultaneously position objects of interest (such as cells of interest) in precisely-defined, clearly-distinguishable locations on the filter membrane 820 (each location corresponding to a single through hole 805). In this non-limiting example, the filter membrane 820 was designed and manufactured to include through holes 805 that are generally about 7 μm in diameter. As will be described in detail below, however, the actual dimensions of a single through hole, such as through hole 805A depicted in FIGS. 8B and 8C, may vary slightly from this target 7 μm dimension. The desired dimension (such as 7 μm in diameter) may also be referred to as a “target dimension” of the through hole, indicative of a minimum allowable dimension of a finished through hole. Thus, the actual, manufactured dimensions of through holes 805 may not be less than the target diameter of 7 μm. Otherwise, the through holes 805 may unintentionally capture objects in a fluid sample that are not objects of interest (for example, an object captured in a through hole 805 that has finished dimensions less than about 7 μm in diameter, even though such object is not of interest). Additionally, the sidewalls of the through holes 805 extending through the interior of filter membrane 820 are advantageously angled or tapered in a manner substantially similar as that described above in reference to FIG. 6A.

Turning now to FIGS. 8B and 8C, close-up images of the through hole 805A of the filter membrane 820 of FIG. 8A are provided. Through hole 805A having tapered sidewalls is representative of the through holes 805 in the filter membrane 820 of FIG. 8A. FIG. 8B is an image of a first opening 850 (e.g., a first opening similar to opening 550 described with reference to FIG. 6) of the through hole 805A taken from a first side 812 of the filter membrane 820 (e.g., a side substantially similar to first side 512 of filter membrane 520 described in reference to FIG. 6A). FIG. 8C is also an image taken from the first side 812 of the filter membrane 820, but is intended to demonstrate the actual dimensions of second opening 860 that is located on the second side 814 of the filter membrane 820.

As discussed above, through hole 805A has a target dimension of 7 μm in diameter (in other words, the filter membrane 820 is designed to include through holes 805 with first openings and second openings that are no less than 7 μm in diameter). As illustrated in the image provided in FIG. 8B, however, the actual diameter of the first opening 850 of the through hole 805 on the first side 812 is greater than the target dimension of 7 μm. For example, the measured diameter of the first opening 550 is 9.067 μm. Additionally, as illustrated in the image provided in FIG. 8C, the actual diameter of the second opening 860 on the second side 814 is greater than the target dimension of 7 μm. For example, the measured diameter of the second opening 560 is 8.268 μm. Further, the inclusion of tapered sidewalls of 805A described above with reference to FIG. 6A is evident from the measurements illustrated in FIGS. 8B and 8C, as the diameter of the first opening 850 of through hole 805A on the first side 812 of filter membrane 820 is larger than the diameter of the second opening 860 of through hole 805A on the second side 814 of filter membrane 820.

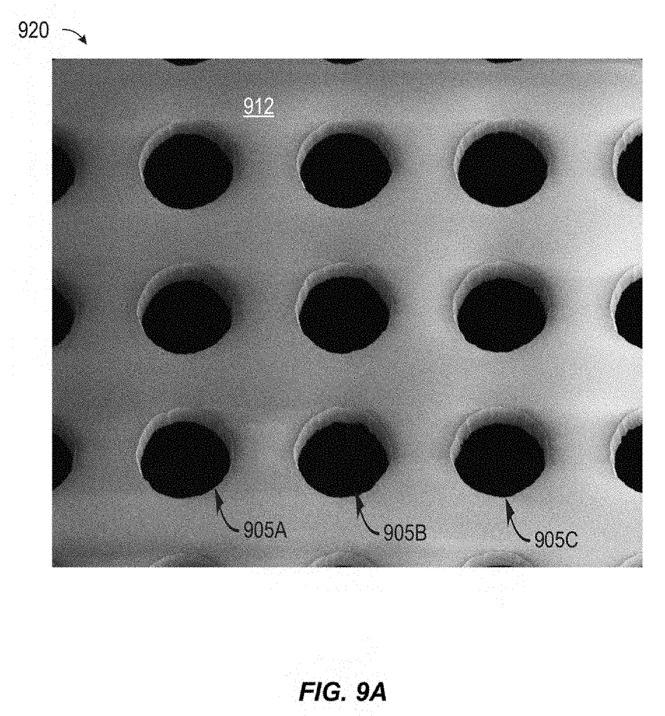

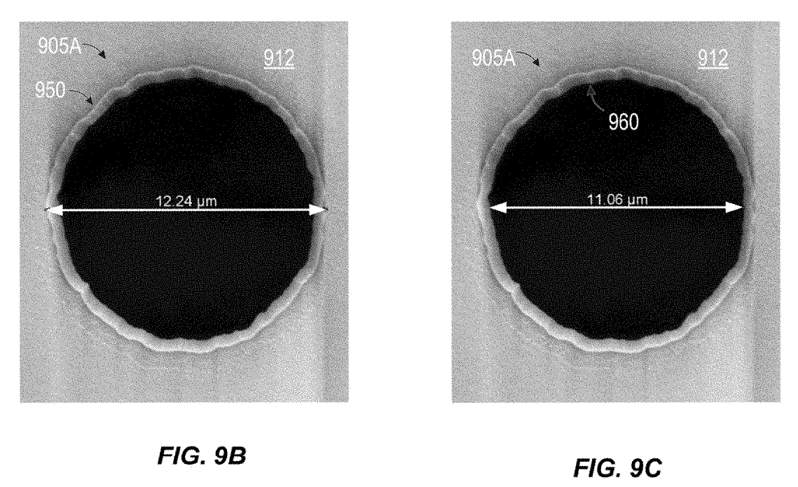

FIG. 9A is an image taken of a portion of a filter membrane 920 having a regular, repeating pattern of through holes 905 according to the present disclosure. FIGS. 9B and 9C are close-up images of a single through hole 905A of the filter membrane 920 of FIG. 9A. Features described above with reference to FIGS. 8A through 8C may apply to the filter membrane 920, with the exception that the through holes 905 have different dimensions than through holes 805 of filter membrane 820. For example, the filter membrane 920 is substantially similar to the filter membrane 820 described with reference to FIGS. 8A through 8C, however the target dimension of the through holes 905 is 10 μm in diameter (rather than 7 μm in diameter). In one non-limiting aspect, the filter membrane 920 is included in a microfluidic chip designed to capture cells of interest that are generally greater than 10 μm in diameter, and to not capture other objects (for example, cells that are not of interest) that are generally smaller than 10 μm in diameter (such as, for example, about 9 μm in diameter). A filter membrane such as filter membrane 720 having circular through holes 705 that are generally 7 μm in diameter would be less suitable for this example implementation, because such a filter membrane would capture both cells of interest (generally greater than 10 μm in diameter) and cells that are not of interest (generally about 9 μm in diameter).

Turning to FIGS. 9B and 9C, close-up images of the through hole 905A of the filter membrane 920 of FIG. 9A are provided. Through hole 905A having tapered sidewalls is representative of the through holes 905 in the filter membrane 920 of FIG. 9A. FIG. 9B is an image of a first opening 950 (e.g., a first opening similar to opening 550 described with reference to FIG. 6A) of the through hole 905A taken from a first side 912 of the filter membrane 920 (e.g., a side substantially similar to first side 512 of filter membrane 520 described with reference to FIG. 6A). FIG. 9C is also an image taken from the first side 912 of the filter membrane 920, but is intended to demonstrate the actual dimensions of second opening 960 that is located on the second side 914 of the filter membrane 820.

As discussed above, through hole 905A has a target height dimension of 10 μm in diameter. As illustrated in the image provided in FIG. 9B, however, the actual diameter of the first opening 950 of the through hole 905 on the first side 912 is greater than the target dimension of 10 μm. For example, the measured diameter of the first opening 950 is 12.24 μm. Additionally, as illustrated in the image provided in FIG. 9C, the actual diameter of the second opening 960 on the second side 914 is greater than the target dimension of 10 μm. For example, the measured diameter of the second opening is 11.06 μm. Further, the inclusion of tapered sidewalls of 905A described above with reference to FIG. 6A are evident from the measurements illustrated in FIGS. 9B and 9C, as the diameter of first opening 950 of through hole 905A on the first side 912 of filter membrane 920 is larger than the diameter of the second opening 960 of through hole 905A on the second side 914 of filter membrane 920.

FIG. 7B illustrates another exemplary microfluidic chip 700B according to one embodiment. The microfluidic chip 700B illustrated in FIG. 7B has circular-shaped through holes, similar in cross-sectional shape to through holes included in microfluidic chip 700A depicted in FIG. 7A. The through holes in microfluidic chip 700B are about 6.5 μm in diameter and are spaced apart by about 0.013 millimeters measured along an x-axis of the microfluidic chip 700B. The microfluidic chip 700B is about 8 millimeters by about 8 millimeters measured along the x-axis and a y-axis of the microfluidic chip, and has an active area that is about 5.1 millimeters by about 5.1 millimeters. The plurality of filters are arranged in a 4×4 grid-like pattern. Each filter is about 1.2 millimeters by about 1.2 millimeters measured along the x-axis and the y-axis of the microfluidic chip. The filters are separated by vanes that are about 0.1 millimeters wide measured along the microfluidic chip. Other configurations are possible.

FIG. 7C illustrates another exemplary microfluidic chip according to one embodiment. The microfluidic chip 700C in FIG. 7C has circular-shaped through holes, similar in cross-sectional shape to through holes in microfluidic chips 700A and 700B. The microfluidic chip 700C is about 8 millimeters by about 8 millimeters measured along the x-axis and y-axis of the microfluidic chip 700C. The filters included in microfluidic chip 700C are manufactured with different dimensions than the filters in the microfluidic chips 700A and 700B. The plurality of filters in microfluidic chip 700C are arranged in a 4×1 grid-like pattern and are about 1.2 millimeters measured along an x-axis of the microfluidic chip by about 5.1 millimeters measured along a y-axis of the microfluidic chip. Other configurations are possible.

### Example 4—Microfluidic Chip with Filter Membrane for Capture of Microbeads

FIG. 10A illustrates an exemplary microfluidic chip 1000 configured to capture and isolate microbeads according to one embodiment. FIG. 10B is a close-up view of one portion of the microfluidic chip 1000. In this non-limiting example, the microfluidic chip 1000 includes a substrate 1010 and a filter membrane 1020. The substrate 1010 includes a frame-shaped exterior portion 1015 and a rectangular-shaped an interior portion 1016. Other configurations are possible. The exterior portion 1015 is about 2 millimeters by about 2 millimeters measured along an x-axis and a y-axis of the microfluidic chip 1000 in this example. The substrate 1010 has a thickness of about 5 μm measured along a z-axis of the microfluidic chip 1000. Other thicknesses are possible. The filter membrane 1020 is disposed on or within the interior portion 1016 in this non-limiting example. The filter membrane 1020 is about 0.48 millimeters by about 0.65 millimeters measured along the x-axis and the y-axis of the microfluidic chip 1000. The filter membrane 1020 in this example thus defines an active area that is about 0.312 millimeters². In this example, the active area of the microfluidic chip 1000 is relatively small, such that the substrate 1010 does not include vanes or any other supporting structure disposed underneath and in contact with the second side of the filter membrane 1020 to provide additional support for the filter membrane 1020.

FIG. 10B illustrates a close-up view of one portion 1011 of filter membrane 1020. The filter membrane 1020 includes circular through holes, such as through hole 1005, arranged in a regular, repeating pattern. However, it will be understood that any filter membrane described herein, not only that illustrated in FIGS. 10A and 10B, may be included in microfluidic chip 1000 depending on the objects sought to be captured, imaged, and analyzed in a particular application. In the embodiment illustrated in FIG. 10, the circular through holes in filter membrane 1020 are generally 5 μm in diameter with a target dimension variation tolerance of about 5%. Through holes having other dimensions are possible as described in detail below.

Each through hole 1005 of filter membrane 1020 is spatially separated from other through holes by a center-to-center pitch of about 10 μm. For example, each grouping of three through holes 1005 form an equilateral triangle with three sides of about 10 μm length connecting the centers of each through hole 1005. The lengths of the sides of this equilateral triangle may be referred to as the “pitch” of through holes 1005. Each through hole 1005 can also be separated or “offset” from adjacent through holes 1005 in the same row by an offset distance 1085 of about 10 μm measured along the x-axis of the filter membrane 1020. The pitch and offset distance can be advantageously selected to maximize the number of through holes in filter membrane 1020 without sacrificing structural integrity of the filter membrane 1020, thus maximizing the number of objects of interest (such as microbeads) that can be captured in the filter membrane 1020. In the embodiment illustrated in FIG. 10, the filter membrane 1020 includes 3,366 circular through holes that are similar to through hole 1005. As described above, these through hole dimensions and spacing are examples and other configurations are possible based on the specific size and shape of the objects (such as microbeads) of interest to be isolated in the microfluidic chip 1000.

The size, shape, and relative spacing of each through hole 1005 in microfluidic chip 1000 can be specifically selected based on the object of interest (such as a microbead) the filter membrane 1020 is designed to capture, such that a single object of interest is captured in each through hole 1005. In other embodiments, the through hole 1005 may have an opening that is generally circular. In one embodiment, filter 1020 comprising circular through holes may be specifically designed and configured to capture microbeads of known characteristics (such as, but not limited to, the size and morphology) of microbeads in a sample. By changing the shape and size of the through holes, multiple filters can be designed and manufactured for the isolation of microbeads having a specific characteristics. Thus, the characteristics and dimensions of each through hole may be specifically selected based on the shape and size of the microbead interest. Further, the density of through holes on a single filter membrane, and the relative position or arrangement of the through holes relative to each other, can be selected to optimize the number of microbeads of interest that are retained or “captured” in the filter membrane.

FIGS. 11A and 11B illustrate an exemplary microfluidic chip 1100 configured to capture and isolate microbeads according to another embodiment. The microfluidic chip 1100 includes a substrate 1110 that is substantially similar to substrate 1010 described with reference to FIG. 10, having an exterior portion 1115 and interior portion 1116 with similar features. In this non-limiting implementation, however, the microfluidic chip 1100 includes a filter membrane 1120 having a plurality of circular through holes 1105 with a target dimension of about 7 μm. The through holes 1105 in this implementation are arranged with a pitch 1180 of about 12 μm. The through holes 1105 are also separated from adjacent through holes 1105 in the same row by an offset distance 1185 of 10 μm measured along an x-axis of the microfluidic chip 1100. Based on this size and spacing of through holes, the filter membrane 1120 includes 2,461 through holes. Additionally, the tolerance for variation in the target dimension is about 5%.

### Example Method of Using a Microfluidic Chip Having a Filter Membrane

FIG. 12 is a flow diagram illustrating on exemplary process 1200 of implementing a microfluidic chip in accordance with embodiments disclosed herein. The process 1200 illustrated at least one method for obtaining cells of interest (such as fetal nucleated RBCs) from a sample using the microfluidic chip disclosed in accordance with the disclosure herein. As illustrated in FIG. 12, the method 1200 can include one or more functions, operations or actions as illustrated by one or more operations 1210-1270.

It is noted that the examples may be described as a process, which is depicted as a flowchart, a flow diagram, a finite state diagram, a structure diagram, or a block diagram. Although a flowchart may describe the operations as a sequential process, many of the operations can be performed in parallel, or concurrently, and the process can be repeated. In addition, the order of the operations may be re-arranged. A process is terminated when its operations are completed. A process may correspond to a method, a function, a procedure, a subroutine, a subprogram, etc. When a process corresponds to a software function, its termination corresponds to a return of the function to the calling function or the main function.

For illustrative purposes, the following description provides for methods for isolation, identification, and harvesting of fetal nucleated RBCs for non-invasive prenatal diagnosis. While the exemplary embodiment disclosed herein may describe isolation of fetal nucleated RBCs from a maternal blood sample for non-invasive prenatal diagnosis, the skilled artisan will understand that the principles and concepts of the methods and devices described herein are applicable beyond NIPT. For example, methods and devices disclosed herein may be configured for the isolation of microbeads, tumor cells for oncology, or any other pathological condition where cells of one kind can be differentiated from cells of another kind based on size, morphology, nuclear staining, and/or biomarker identification.

Embodiments of methods and devices disclosed herein can obtain cells of interest using morphology-based isolation combined with affinity and/or biomarker-based detection and identification. By combining these processes on an integrated microfluidic chip in accordance with the embodiments described herein, the method 1210 resolve long-standing challenges associated with isolating specific cells of interest from a sample of cells. Unlike fluorescence-activated cell sorting (“FACS”) utilized in flow cytometry, embodiments disclosed with reference to method 1200 are visualization-based methods similar to imaging cytometry that are performed on a microscope platform, but advantageously address drawbacks associated with prior imaging cytometry-based systems and methods. Method 1200 can be partially or fully automated which adds another benefit to embodiments described in the current disclosure.

Cytometry, including flow cytometry and imaging cytometry, is the measurement and/or identification of cell characteristics. Cytometry methodologies are configured to measure any of a number of parameters, including for example cell size, cell count, cell shape and structure, cell cycle phase, DNA content, and the existence or absence of specific proteins on the cell surface or within the cell. There are many applications in which the different cytometry methods can be used. For example, cytometry can be used in characterizing and counting blood cells in a sample of blood, cell biology research, and medical diagnostics to characterize cells in pathological diseases (e.g., cancer and AIDS). Imaging cytometry is one type of cytometry that operates by statically imaging a large number of cells using optical microscopy. Prior to analysis, cells can be stained to enhance contrast or detect specific molecules by labeling these with nuclear stains, biomarkers, and/or fluorescent dyes.

One non-limiting advantage of embodiments of microfluidic chips disclosed herein is that it can be used in imaging cytometry to advantageously develop a representation (for example, obtain an image or take a picture) of all of the captured cells in a specific area of interest in a single image. In one non-limiting aspect, the specific area of interest is one region of a plurality of regions of a single filter membrane of a microfluidic chip. In another non-limiting aspect, the specific area of interest is one filter membrane arranged in a microfluidic chip including one single filter membrane. In another example, the specific area of interest is one filter membrane of a plurality of filter membranes arranged in a microfluidic chip. Due to the precisely-defined and repeating grid pattern of through holes in the filter membrane(s), the exact position of each captured and hydrodynamically retained cell can be identified using the unique position of its corresponding through hole in the filter membrane(s). In one embodiment, capturing and simultaneously positioning of cells of interest in this way allows the cells of interest to be analyzed for verification that the captured cells are, in fact, cells of interest. For example, where the cell samples have been stained and/or labeled with nuclear stain, specific biomarkers, and/or fluorescent dyes used to identify user-defined characteristics of the cells, captured cells with those characteristics may be readily identified and their position can be easily returned to for subsequent, more detailed analysis of a captured cell or for manipulation or extraction of a captured cell. In another embodiment, the capturing and simultaneously positioning of cells of interest in this way allows the cells of interest to be subject to steps of cell lysis and DNA extraction for downstream genetic analysis. For example, the captured, isolated, and sorted cells of interest can be assessed for a nucleotide sequence of nucleic acid molecules or expression of a gene.

Embodiments of filters described herein can advantageously be used to distinguish captured cells of interest from captured cells that are not of interest based on a second criterion: biomarkers specific to the cells of interest (in this non-limiting example, fetal nucleated RBCs). For example, before or after the sample is run through the filter and cells are captured in the filter membrane(s), the cells can be stained and/or labeled with nuclear stain, specific biomarkers, and/or fluorescent dyes for positive or negative selection of a subset of the captured cells (for example, positive selection of captured fetal nucleated RBCs and negative selection of captured cells that are not fetal nucleated RBCs).

As used herein, “microscope platform” refers to a system and/or device configured to perform imaging of cells. In one aspect, a microscope platform includes an epifluorescence microscope. The microscope platform may include an imaging device configured with an adjustable or multiple magnification objective (e.g., 10×, 40×, 60×, etc.), and an image sensor configured to obtain an image based on the light received through an imaging device lens. In some embodiments, the imaging device includes a field-of-view (“FOV”) that is configured to match the size and shape of at least one region of a filter membrane of the microfluidic chip as defined by vanes of a substrate that support the filter membrane. In some embodiments, the microscope platform may be configured to scan along a microfluidic filter membrane including a plurality of filter regions and obtain at least one image of each filter region, where the dimension of each filter region corresponds to the FOV of the imaging device.

Method 1200 can begin at operation 1210, “Providing a sample.” Operation 1210 can be followed by operation 1220, “Applying the sample to a filter membrane integrated on a microfluidic chip.” Operation 1220 can be followed by operation 1230, “Labeling cells in the sample.” Operation 1230 can be followed by operation 1240, “Isolating cells of interest in the sample.” In some cases, operation 1220 and operation 1240 are performed simultaneously. Operation 1240 can be followed by operation 1250, “Imaging cells captured in the filter membrane.” Operation 1250 can be followed by an optional operation 1260, “Removing cells of no interest.” The method next moves to operation 1270, “Harvesting confirmed cells of interest.”

At operation 1210, “Providing a sample,” a sample containing cells of interest may be provided. For example, maternal samples containing one or more fetal nucleated cells, such a red blood cell, can be obtained from human pregnant mothers using standard blood draw. The maternal sample can be taken during the first trimester (about the first three months of pregnancy), the 2nd trimester (about months 4-6 of pregnancy), or the third trimester (about months 7-9 of pregnancy). In some embodiments, a blood sample is obtained from a pregnant human mother even after a pregnancy has terminated. Typically, the sample obtained is a blood sample.

At operation 1220, “Applying the sample to a filter membrane integrated on a microfluidic chip,” embodiments of microfluidic chips having filter membranes described herein that are suitable to select fetal nucleated blood cells may be used. In some embodiments, the microfluidic chip and filter membrane used in this non-limiting example are substantially similar to the microfluidic chip depicted in FIGS. 1A through 11B. Accordingly, in some embodiments, fetal nucleated RBCs may be captured when mature RBCs pass through filter holes having a size and/or shape that allow mature RBCs to pass through, but not fetal nucleated RBCs.

In some embodiments, a filter membrane may be coated with a binding moiety or affinity molecule that selectively binds fetal nucleated cells, such as fetal nucleated RBCs. For example, an antibody that specifically binds to fetal nucleated RBCs may be used to coat the filter membrane, so that fetal nucleated RBCs are retained while the mature RBCs pass through the filter membrane.

In some embodiments, a sample applied to a filter membrane at operation 1220, can be dominated (>50%) by cells not of interest (e.g., nucleated maternal red blood cells). In some cases, the nucleated fetal cells of a sample applied to the filter membrane make up at least 2, 3, 4, 5, 10, 20, 30, 40, 50, 60, 70, 80, 90 or 95% of all cells in the sample. In some embodiments, the use of embodiments of microfluidic chips disclosed herein have removed at least 50, 60, 70, 80, 85, 90, 91, 92, 93, 94, 95, 96, 97, 98, 99, 99.5, 99.6, 99.7, 99.8 or 99.9% of all unwanted analytes (e.g., maternal cells such as platelets and leukocytes, mature RBCs) from a sample.

At operation 1230, “Labeling cells in the sample,” cells may be labeled, directly or indirectly, with a dye in a staining process. Any fluorescent dye that is used in fluorescence microscopy can be used. For example, the nucleated fetal RBCs may be labeled, directly or indirectly, with a dye indicative of certain characteristics of the cell. In some embodiments, the labeling procedure of operation 1230 may be performed prior to, during, or after operation 1220. In some embodiments, a dye that stains DNA, such as Acridine orange (AO), ethidium bromide, hematoxylin, Nile blue, Hoechst, Safranin, or DAPI, may be used. In some embodiments, a cell type-specific dye, for example, a dye that specifically labels a fetal cell or a non-fetal cell, may be used. The cell type-specific dye may be used to label the cells directly or indirectly, for example, through a cell type-specific antibody. The labeling strategy involved may be sequentially carried out or simultaneously carried out.

Any of a variety of fluorescent molecules or dyes can be used to label cells in methods provided herein, including, but not limited to, Alexa Fluor 350, AMCA, Alexa Fluor 488, Fluorescein isothiocyanate (FITC), GFP, RFP, YFP, BFP, CFSE, CFDA-SE, DyLight 288, SpectrumGreen, Alexa Fluor 532, Rhodamine, Rhodamine 6G, Alexa Fluor 546, Cy3 dye, tetramethylrhodamine (TRITC), SpectrumOrange, Alexa Fluor 555, Alexa Fluor 568, Lissamine rhodamine B dye, Alexa Fluor 594, Texas Red dye, SpectrumRed, Alexa Fluor 647, Cy5 dye, Alexa Fluor 660, Cy5.5 dye, Alexa Fluor 680, Phycoerythrin (PE), Propidium iodide (PI), Peridinin chlorophyll protein (PerCP), PE-Alexa Fluor 700, PE-Cy5 (TRI-COLOR), PE-Alexa Fluor 750, PE-Cy7, APC, APC-Cy7, Draq-5, Pacific Orange, Amine Aqua, Pacific Blue, Alexa Fluor 405, Alexa Fluor 430, Alexa Fluor 500, Alexa Fluor 514, Alexa Fluor-555, Alexa fluor-568, Alexa Fluor-610, Alexa Fluor-633, DyLight 405, DyLight 488, DyLight 549, DyLight 594, DyLight 633, DyLight 649, DyLight 680, DyLight 750, or DyLight 800. Such fluorescent molecules or dyes may produce a corresponding light signature or spectrum upon being illuminated or excited by a light source having a particularly corresponding wavelength of light. Thus, using specific fluorescent molecules or dyes may produce an indicator of a particular nucleic acid, antibody or antibody-based fragment probe present on or within a fetal cell.

In some embodiments fetal biomarkers can be used to label one or more fetal cells at operation 1230 of FIG. 1200. For example, this can be performed by distinguishing between fetal and maternal cells based on relative expression of a gene (e.g., DYS1, DYZ, CD-71, MMP14) that is differentially expressed during fetal development. In one embodiment of the present disclosure, detection of transcript or protein expression of one or more genes including, MMP14, CD71, GPA, HLA-G, EGFR, CD36, CD34, HbF, HAE 9, FB3-2, H3-3, erythropoietin receptor, HBE, AFP, APOC3, SERPINCI, AMBP, CPB2, ITIH1, APOH, HPX, beta-hCG, AHSG, APOB, J42-4-d, 2,3-biophosphoglycerate (BPG), Carbonic anhydrase (CA), or Thymidine kinase (TK), is used to enrich, purify, enumerate, identify, detect, or distinguish a fetal cell. The expression can include a transcript expressed from these genes or a protein. In one embodiment of the present disclosure, expression of one or more genes including MMP14, CD71, GPA, HLA-G, EGFR, CD36, CD34, HbF, HAE 9, FB3-2, H3-3, erythropoietin receptor, HBE, AFP, AHSG, J42-4-d, BPG, CA, or TK, is used to identify, purify, enrich, or enumerate a fetal nucleated cell such as a fetal nucleated RBC.

In another embodiment of the present disclosure, fetal cells known as trophoblasts are a cell of interest that is isolated using filters described herein. Biomarkers specific to trophoblasts can be labeled and used to distinguish the fetal trophoblast cells (that are captured in the filter and are objects of interest) from maternal cells (that are also captured in the filter but are not objects of interest). Biomarkers that can be used to label, identify, detect, or distinguish a fetal trophoblast cell include (but are not limited to) cytokeratin 5, 6, 7, 8, 10, 13, 14, 18, 19; CD147, CD47, CD105, CD141, CD9, HAI-1, CD133, HLA-G, human placental lactogen, PAI-1, and IL-35. Other biomarkers that are not specific to fetal trophoblast cells but that can be used to label, identify, detect, or distinguish fetal cells of interest from maternal cells that are not of interest, include (but are not limited to) CD68, CD105, placental alkaline phosphatase (PLAP), NDOG, GB25, β-hCG, and 3b-hydroxy-5-ene steroid dehydrogenase. The above-referenced list of biomarkers provides examples of suitable biomarkers for labeling, identifying, detecting, or distinguishing a fetal cell from a maternal cell and is not intended to limit methods and devices described herein, which can capture and identify any cell of interest that is subject to filtration, whether or not the cell of interest has biomarkers that are used to distinguish the cell of interest captured in the filter from another object that is also captured in the filter but is not a cell of interest.

At operation 1240, “Isolating cells of interest in the sample,” cells of interest such as fetal cells may be isolated using embodiments of microfluidic chips and filter membranes described herein with reference to FIGS. 1A through 11B. Isolating cells of interest can include positioning a single cell of interest at a distinct, precisely-defined location, such as a single through hole, in a filter membrane. As described above, each fetal nucleated RBC may be isolated from other cells in the sample (other fetal nucleated RBCs, non-nucleated fetal cells, maternal cells, etc.) when the fetal nucleated RBC is retained in a single through hole of the filter membrane while other cells that are not of interest (such as mature maternal RBCs) pass through the through holes of the filter membrane and are not retained in the filter membrane. Accordingly, the isolation operation 1240 may be performed at the same time as operation 1220.

In operation 1250, “Imaging cells captured in the filter membrane” the cells captured in the filter at operation 1220 and/or operation 1240 are imaged for further analysis and genetic testing downstream. In some embodiments, imaging at operation 1250 also includes imaging each filter region of a plurality of filter regions of a filter membrane using a microscope platform with a field of view (FOV) that matches the dimensions of a single filter region as defined by vanes of a substrate in the microfluidic chip, as described in reference to FIGS. 1A through 3 and 7. In some embodiments, the FOV is defined by the filter region and filter membrane where vanes are omitted as described in reference to FIGS. 10A through 11B.

In some embodiments, cell samples are labeled or stained with fluorophores, fluorescent chemical compounds that can re-emit light upon light excitation. Cells samples can be labeled or stained with multiple kinds of fluorophores, each kind designed to emit a specific color of light upon light excitation. Embodiments of the microscope platform include an illumination source configured to illuminate fluorescently-stained cells in a filter with light of a specific wavelength which is absorbed by the fluorophores, causing them to emit light of longer wavelengths (i.e, of a different color than the absorbed light). The specific wavelength can be selected based on the nuclear staining and/or biomarker identification used to fluorescently stain the cell sample. In some embodiments, the microscope platform further includes a detector or a sensor configured to detect the spectral emission characteristics of the fluorophore used to label the fluorescently-stained cell. The distribution of a single fluorophore (color) can be imaged by the microscope platform. Multi-color images of several kinds of fluorophores can be developed using several single-color images. In one embodiment, the microscope platform is configured to have multiple illumination sources or modify the illumination of the captured cells to cause fluorescence of multiple different dyes.

For example, FIGS. 14A and 14B illustrate images taken of one filter membrane of a microfluidic chip used to capture cells of interest in through holes according to the present disclosure. These figures illustrate images taken during one implementation of the method 1200 performed to detect and identify fetal nucleated RBCs from a maternal blood sample using a filter membrane. FIGS. 14A and 14B depict a filter membrane that may be substantially similar to the one of the filter membranes of the microfluidic chips described herein with reference to FIGS. 1A through 11B. FIGS. 14A and 14B are actual images of a filter membrane taken by a microscope platform, where cells of interest in a sample or portions of the sample are captured and retained in precisely-defined and identifiable through holes on the filter. FIG. 14A shows unstained cells of interest in brightfield. FIG. 14B shows cells of interested that have been labeled or stained in accordance with the disclosure herein.

At operation 1270, “Harvesting confirmed cells of interest,” the cells of interest are removed from the filter membrane for genetic and/or diagnostic analysis. For example, cells identified as cells of interest at operation 1250 are next harvested at operation 1270. In some embodiments, a micromanipulator may be used to harvest and/or pluck cells of interest from the through holes during operation 1270. For example, a micromanipulator may include a needle configured to pluck cells captured in each through hole of the filter membrane. The needle tip and movement can be engineered so as not to puncture the filter membrane. The insertion and removal of the needle in each through hole may exert an outward force onto the sidewalls of a given through hole, thus the material, dimensions, and through hole density of the filter membrane can be selected to withstand this force such that the filter membrane does not break or the through hole is not deformed. In some cases, these advantageous mechanical properties of the filter membrane allow for a user to repeatedly use the same filter membrane to process a single sample, for example, by apply additional portions of the sample to the filter membrane after operation 1250 and before harvesting all captured cells of interest at operation 1270.

In some embodiments, cells not of interest may be isolated in operation 1240. Where captured cells are confirmed to be cells that are not of interest, operation 1260 may optionally be performed to destroy, fragment, and/or remove the captured cell from its respective through hole, thereby permitting capture of cells of interest in the now-cleared through hole that was previously occupied by the cell not of interest. In one non-limiting embodiment, operations 1210-1250 can be repeated after cells that are not of interest are removed during operation 1260. Repeating method 1200, or certain operations in method 1200, in this manner can result in a microfluidic chip having a large number of cells of interest captured in the filter membrane(s) of the microfluidic chip. Microfluidic chips with a maximum density of cells of interest can thus be obtained by repeating method 1200, or certain operations of method 1200, on the same microfluidic chip after cells not of interest are removed at each iteration of operation 1260. In some aspects, harvesting of confirmed cells of interest in operation 1270 is only performed after a significant number of through holes have captured confirmed cells of interest. Thus, the distinct, precisely-defined position of each through hole within the microfluidic chip enables the extraction and/or manipulation of captured cells that are of interest, as well as cells that are not of interest.

### Example Method of Fabricating a Microfluidic Chip with Filter Membrane Having Through Holes

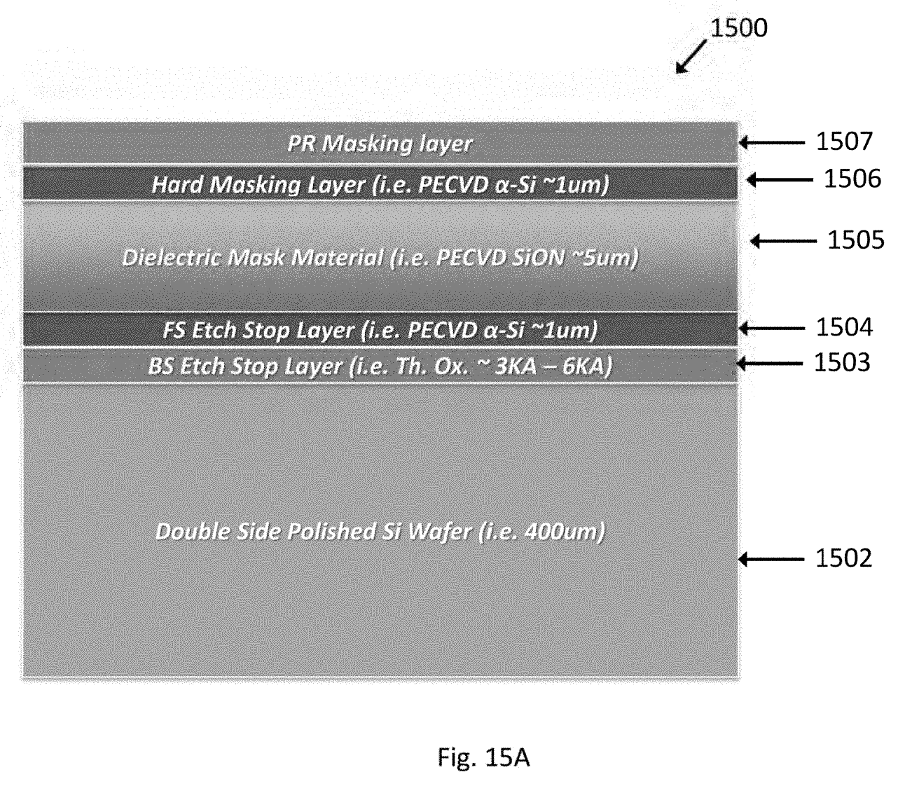

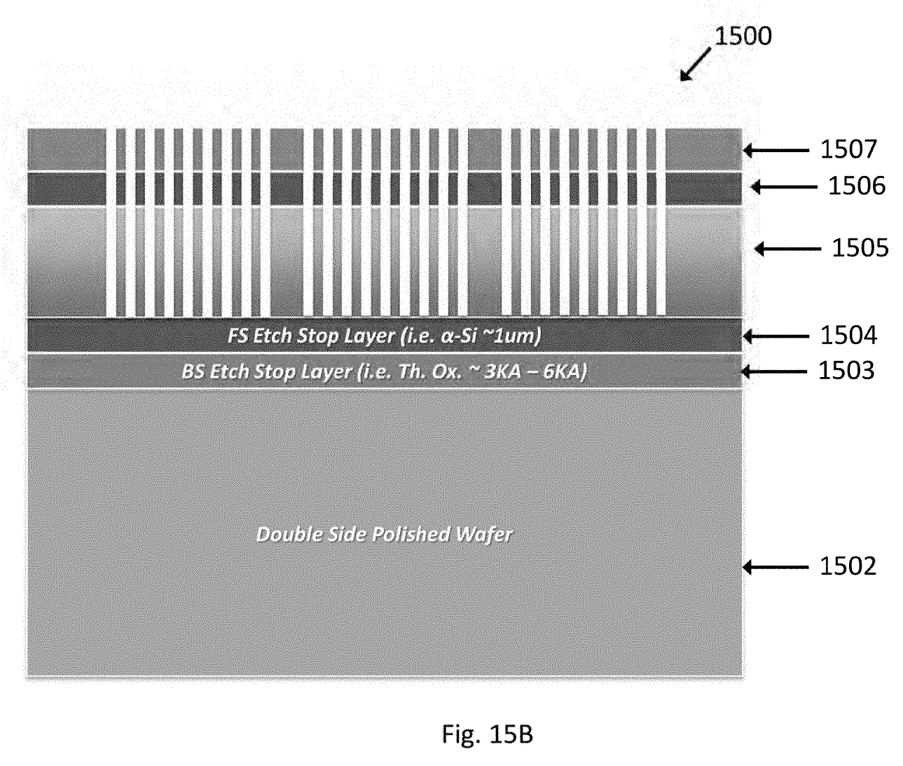

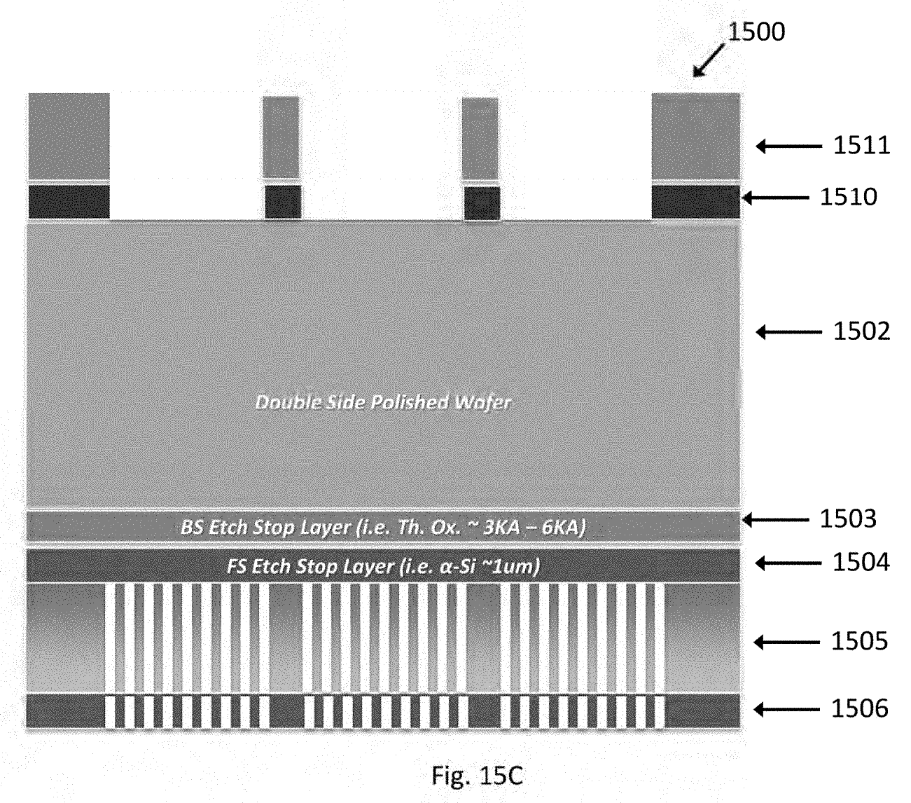

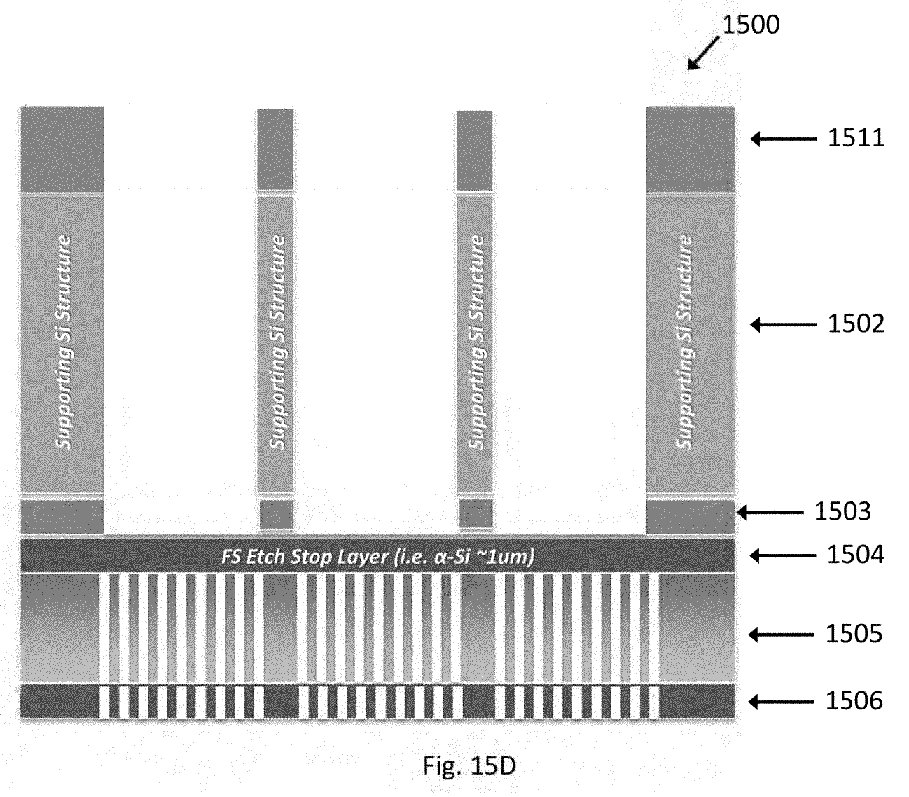

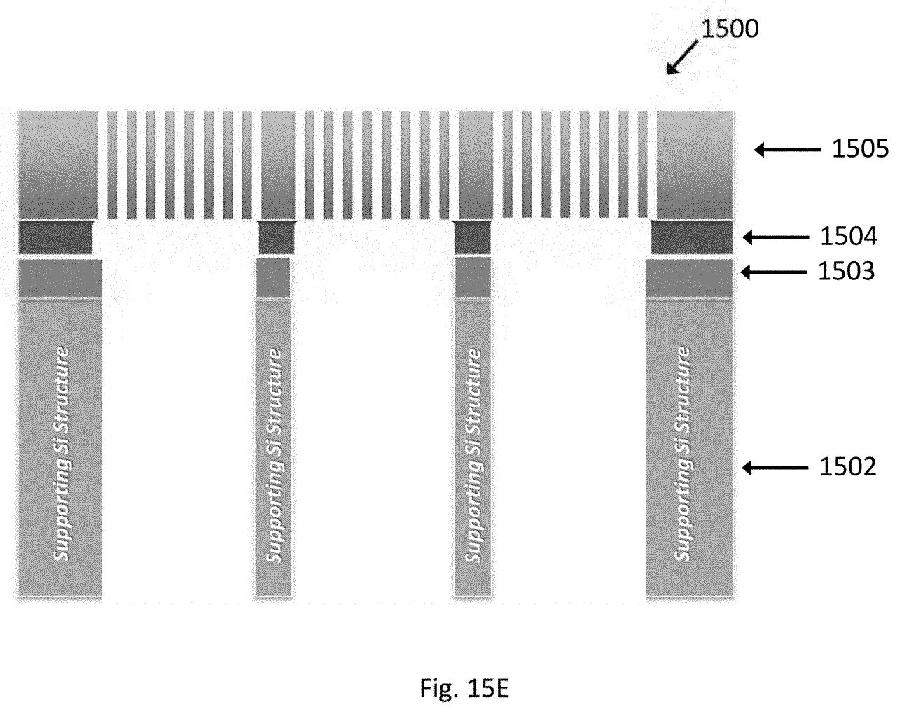

FIGS. 15A through 15E show example cross-sectional views of schematic illustrates of an example fabrication process of fabricating a microfluidic chip as described herein.

While the shapes and dimensions of the respective filter regions and through holes may differ from the non-limiting examples illustrated in FIGS. 15A through 15E, methods of fabricating embodiments of microfluidic chips described herein involve similar features. Therefore, the following describes a method of fabricating a microfluidic chip having a filter region and through holes with reference to the microfluidic chip illustrated in FIGS. 15A through 15E, but it will be understood that the same or a substantially similar process may be performed to develop a microfluidic chip having differently-shaped and differently dimensioned filter regions and through holes, for example, such as those described above with reference to FIGS. 1A through 11B. Additionally, the steps illustrated in FIGS. 15A through 15E are preferably performed in the illustrated order; however, as will be understood by those skilled in the art, they may also be performed in other sequences and various substitutions and replacements may be made. In the discussion below, some of the possible substitutions and replacements will be discussed in further detail. Further, while omitted in the following description, appropriate cleaning steps can be performed periodically and as needed to prepare a given layer for a following processing step and/or to clean the layer based on the previously processed step.

As used herein, the term “wafer” will be used to describe an incomplete microfluidic chip, and the term “microfluidic chip” will be used to describe the completed integrated microfluidic chip. For example, FIGS. 15A through 15E each illustrate one embodiment of a stage of fabricating an integrated microfluidic chip, where wafer 1500 refers to each stage in the process. For example, FIG. 1A illustrates one embodiment of the completed microfluidic chip 100 fabricated using the process described herein, where each of FIGS. 15A through 15E represents at least one stage of the fabrication process that concludes with the microfluidic chip 100 of FIG. 1A.

The process begins where a substrate 1502 is provided as shown in FIG. 15A. The substrate 1502 can be formed of any suitable material and have any suitable dimension to support the filter membrane formed later in the process. In some cases, the substrate 1502 is silicon wafer. The silicon wafer can be a commercially available, conventionally-sized wafer that is processed to obtain desired dimensions for the substrate 1502. For example, the substrate 1502 can be thinned down to have a thickness of approximately 400 microns. The thickness of the substrate 1502 can be selected based on the needs of the particular application for which the microfluidic chip is intended. In some embodiments, the substrate 1502 may be a solid or semi-solid substrate that may be planar in structure, i.e., substantially flat or having at least one substantially flat surface. In some embodiments, the substrate 1502 may be a double-sided polished silicon wafer having two surfaces polished to a clean and flat surface for processing. The planar substrate can be manufactured using solid substrates common in the fields of microfabrication, for example, silica-based substrates, such as glass, quartz, silicon or polysilicon, as well as other known substrates, for example, gallium arsenide. In the case of these substrates, common microfabrication techniques, such as photolithographic techniques, wet chemical etching, micromachining (drilling, milling and the like), may be readily applied in the fabrication of microfluidic chips and substrates. Alternatively, polymeric substrate materials may be used to fabricate the devices of the present disclosure, including, for example, polydimethylsiloxanes (PDMS), polymethylmethacrylate (PMMA), polyurethane, polyvinylchloride (PVC), polystyrene, polysulfone, polycarbonate, and the like. In the case of such polymeric materials, injection molding or embossing methods may be used to form the substrates. In such cases, original molds may be fabricated using any of the above described materials and methods. The assembled microfluidic chips may be treated with plasma to alter surface wet-ability where desired post assembly or preferably treated first and then assembled.

The process continues where a backside (BS) etch stop layer 1503 and a frontside (FS) etch stop layer 1504 is formed. Formation of the BS etch stop layer 1503 and FS etch stop layer 1504 may be performed by with the depositing the respectively layers onto the substrate 1502. The BS etch stop layer 1503 and FS etch stop layer 1504 can be formed of any suitable material having the sought after properties. Exemplary materials for the BS etch stop layer 1503 may include thermal oxide or other materials exhibiting similar properties. In some embodiments, the BS etch stop layer 1503 may have a thickness of approximately 3000 angstroms to 6000 angstroms. Other configurations are possible. Exemplary materials for the FS etch stop layer 1504 may include amorphous silicon (α-Si) or other materials exhibiting similar properties. In some embodiments, the FS etch stop layer 1504 may have a thickness of approximately 1 micron. Other configurations are possible. Deposition of the BS and FS etch stop layer materials may be carried out using deposition techniques such as physical vapor deposition (PVD, e.g., sputtering), plasma-enhanced chemical vapor deposition (PECVD), thermal chemical vapor deposition (thermal CVD), E-Beam evaporation, or spin-coating a thin layer of the selected material onto substrate 1502. In some embodiments, the BS etch stop layer 1503 and FS etch stop layer 1504 may be a single etch stop layer.

The process continues where a dielectric mask material layer 1505 is formed. Formation of the dielectric mask material layer 1505 may be performed by depositing the layer onto the FS etch stop layer 1504. The dielectric mask material layer 1505 may represent the filter membrane material. The dielectric mask material layer 1505 can be formed of any suitable material having the sought after properties. Exemplary materials for the dielectric mask material layer 1505 may include silicon oxynitride or other materials exhibiting similar properties. For example, the materials may be selected to have neutral stress properties, visually transparent, low intrinsic florescence, electrically inert, and etch resistant. In some embodiments, the dielectric mask material layer 1505 may have a thickness of approximately 5 microns. Other configurations are possible. Deposition of the dielectric mask material layer 1505 may be carried out using deposition techniques such as physical vapor deposition (PVD, e.g., sputtering), plasma-enhanced chemical vapor deposition (PECVD), thermal chemical vapor deposition (thermal CVD), E-Beam evaporation, or spin-coating a thin layer of the selected material onto FS etch stop layer 1504.

The process continues where through holes are defined in the filter membrane based on a hard mask layer 1506 and a photoresist masking layer 1507. In some embodiments, a photoresist layer alone is sufficient as a patterning layer, thus the hard mask layer 1506 need not be included. This may be based on the etch performance of the photoresist and/or hard mask layer as relative to the layer to be etched (e.g., the layers on which the patterning layers are deposited). For example, where the etch performance of the dielectric mask material layer 1505 is greater than the photoresist layer 1507, than a hard mask layer 1506 may be required.

Thus, in one example, the process continues where a hard mask layer 1506 is formed. Formation of the hard mask layer 1506 may be performed by with the depositing the layer onto the dielectric mask material layer 1505. The hard mask layer 1506 can be formed of any suitable material having the sought after properties, for example, metallic, organic, or inorganic materials. Exemplary materials for the hard mask layer 1506 may include α-Si or other materials exhibiting similar properties. In some embodiments, the hard mask layer 1506 may have a thickness of approximately 1 micron. Other configurations are possible. Deposition of the hard mask layer 1506 material may be carried out using deposition techniques such as physical vapor deposition (PVD, e.g., sputtering), plasma-enhanced chemical vapor deposition (PECVD), thermal chemical vapor deposition (thermal CVD), E-Beam evaporation, or spin-coating a thin layer of the selected material onto dielectric mask material layer 1505.

The process continues where through holes are defined in a photoresist layer 1507 including a pattern corresponding to the desired through hole size, layout, and arrangement. The photoresist layer 1507 may be deposited onto hard mask layer 1506 and patterned using conventional lithographic techniques (such as, but not limited to, hard mask, photoresist, exposure, development, and other suitable techniques). In one example embodiment using photoresist 1507 and/or hard mask layer 1506 to define through holes pattern in the dielectric mask material layer 1505, a photoresist layer 1507 is deposited through PVD, PECVD, thermal CVD, or spin-coating onto the hard mask layer 1506.

The photoresist 1507 and hard mask layer 1506 are configured to permit exposure of the areas of the dielectric material layer 1505 intended to be removed, thereby leaving the material of the dielectric material layer 1505 defining the through holes of the filter membrane. The wafer 1500 is then exposed to light which causes a chemical change such that the exposed regions of the photoresist layer 1506 and hard mask layer 1507 are removed by a development step. The development step is performed by applying a developing solution to the surface of the microfluidic chip, the solution being configured to remove the exposed regions of the photoresist layer 1507 and hard mask layer 1506, as illustrated in FIG. 15B.

The process continues to FIG. 15B where the through holes are patterned the photoresist layer 1507 using conventional lithographic techniques (such as, but not limited to, hard mask, photoresist, exposure, development, and other suitable techniques) onto FS etch stop layer 1504. For example, in one embodiment, deep reactive ion etching (DRIE) may be used to pattern the photoresist layer 1507 onto the hard mask layer 1506. Then, the combination of the photoresist layer 1507 and hard mask layer 1506 may be used to pattern the through hole pattern onto the dielectric material layer 1505 via DRIE. The FS etch stop layer 1504 may be configured to stop the DRIE processing using optical end points techniques. In some embodiments, the remaining photoresist layer 1507 may be removed following etching the hard mask layer 1506, and then use the hard mask layer 1506 alone as a patterning material to pattern the dielectric material layer 1505 using the DRIE. Once the dielectric material layer 1505 is etched, the photoresist layer 1507 is removed using conventional processing techniques.

The process continues with the processing of the backside of the substrate 1502 as illustrated in FIG. 15C. FIG. 15C illustrates partial cross-sectional side views of wafer 1500 having been flipped 180 degrees relative to the illustration of wafer 1500 in FIGS. 15A and 15B. In some embodiments, appropriate cleaning steps can be performed to the backside of substrate 1502 to prepare the backside surface for a following processing step. Once the wafer 1500 is flipped, BS etch stop layer 1503 and FS etch stop layer 1504 are aligned with patterns and deposition layers to be applied subsequently.

The process continues where support structures and vanes are defined in the substrate based on a hard mask layer 1510 and a photoresist masking layer 1511 deposited on the backside of substrate 1502. In some embodiments, a photoresist layer alone is sufficient as a patterning layer, thus the hard mask layer 1506 need not be included. This may be based on the through silicon via (TSV) etch characteristics and selectivity's of the photoresist and/or hard mask layer as relative to the substrate to be etched. For example, where the etch performance of the substrate 1502 is greater than the photoresist layer 1511, than a hard mask layer 1510 may be required.

Thus, in one example, the process continues where a hard mask layer 1510 is formed on the backside of substrate 1502. Formation of the hard mask layer 1510 may be performed in a manner similar to depositing hard mask layer 1506. The hard mask layer 1510 can be formed of a material that is similar or the same as hard mask layer 1506. Exemplary materials for the hard mask layer 1510 may include α-Si or other materials exhibiting similar properties. In some embodiments, the hard mask layer 1510 may have a thickness of approximately 1 micron. Other configurations are possible.

The process continues where the support structure and vanes are defined in a photoresist layer 1511. The photoresist layer 1511 may be deposited onto hard mask layer 1510 and patterned using conventional lithographic techniques (such as, but not limited to, hard mask, photoresist, exposure, development, and other suitable techniques). The photoresist layer 1511 may be deposited through PVD, PECVD, thermal CVD, or spin-coating onto the hard mask layer 1510.

Similar to that described above, the photoresist 1511 and hard mask layer 1510 are configured to permit exposure of the areas of the substrate 1502 intended to be removed, thereby leaving the material of the substrate 1502 defining the support structure and vanes of the microfluidic chip. The wafer 1500 is then exposed to light which causes a chemical change such that the exposed regions of the photoresist layer 1511 and hard mask layer 1510 are removed by a development step. The development step is performed by applying a developing solution to the surface of the microfluidic chip, the solution being configured to remove the exposed regions of the photoresist layer 1511 and hard mask layer 1510, as illustrated in FIG. 15C.

The process continues to FIG. 15D where the support structures and vanes are patterned in the photoresist layer 1511 using conventional lithographic techniques onto FS etch stop layer 1504. FIG. 15D illustrates an embodiment where the hard mask layer 1510 is not used and only photoresist layer 1511 is used to pattern the support structure and vanes into substrate 1502. For example, in one embodiment, support structures and vanes may be patterned into the substrate 1502. The DRIE TSV process may be stopped at the BS etch stop layer 1503 (not shown). Then, using the same or a subsequent etching process (e.g., DRIE TSV) the BS etch stop layer 1503 may be etched and stopped at the FS etch stop layer 1504, thereby patterning the support structure and vanes onto the FS etch stop layer 1504.

The process continues to as illustrated in FIG. 15E, where the photoresist layer 1511 (and hard mask layer 1510 if present) is removed through conventional processing techniques. In some embodiments, appropriate cleaning steps can be performed to the backside and frontside of substrate 1502 to prepare the wafer 1500 for further processing. In some embodiments, the hard mask layer 1506, if present, is etched using wet etching techniques (not shown). In some embodiments, the FS etch stop layer 1504 is etched using wet etching techniques (not shown). In embodiments where the hard mask layer 1506 and FS etch stop layer 1504 are the same material (e.g., α-Si), the wet etching technique may utilize potassium hydroxide (KOH) solutions.

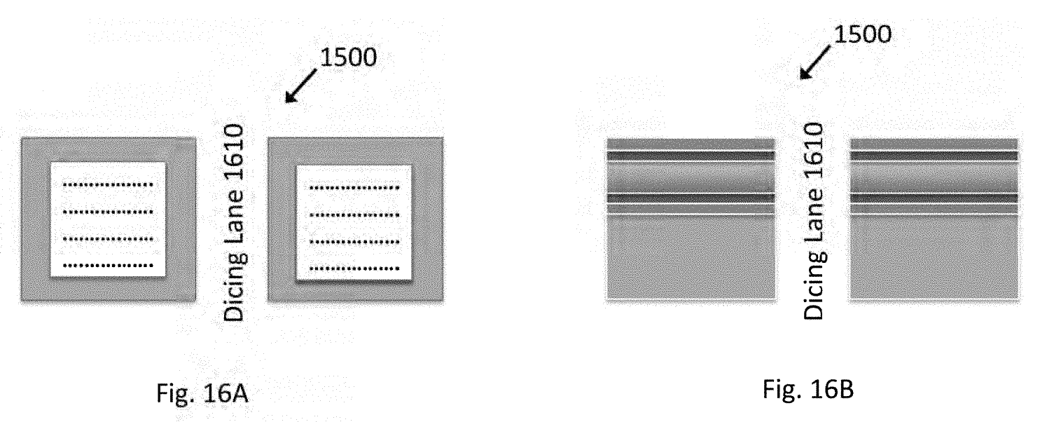

Once the wafer 1500 is cleaned and processed, the wafer 1500 may be diced into individual microfluidic chips as described herein. For example, FIGS. 16A and 16B illustrate dicing lanes. FIG. 16A illustrates a top down view of wafer 1500 comprising two microfluidic chips diced along the dicing lane 1610. FIG. 16B illustrates a cross-section view of wafer 1500 comprising two microfluidic chips diced along the dicing lane 1610. In some embodiments, hard mask layer 1506 can be exposed in dicing lanes while patterning hard mask layer 1506 via photoresist layer 1507, which may remove hard mask layer 1506 remaining in the dicing lanes while etching the hard mask layer 1507. In another embodiment, the filter membrane (e.g., dielectric mask material layer 1505) may be exposed in dicing lanes while patterning the through holes, which may remove dielectric mask material layer 1505 material remaining in dicing lanes. In another embodiment, the dicing lanes may be exposed during baskside patterning (e.g., FIG. 15D), which will also pattern dicing lanes through substrate 1502 during the backside etching. This embodiment may remove the need for a dicing step.

In another embodiment, the dicing step may be performed by stealth dicing techniques, which may be done on the frontside or backside of wafer 1500. This embodiment may remove the need for allocating additional space on wafer 1500 for dicing lanes. Some non-limiting advantages of stealth dicing are that the technique is vibrationless with no impact, and no substrate material is lost. Also, the dry and cleaning process in and out reduces the chance of damage to the filter membrane, through hole contamination, clogging, or need for post dicing cleaning.

In another embodiment, photoresist layer 1506 is deposited on dielectric mask material layer 1505 which is also deposited on a single etch stop layer (e.g., FS etch stop layer 1504 and BS etch stop layer 1503 are combined to a single layer). These layers are all deposited on the substrate 1502. In this embodiment, the backside of substrate 1502 may be patterned and etched in a first processing step via TSV. The frontside of substrate 1502 may then be patterned and etched through the dielectric mask material layer 1505 and etch stop layer using the photoresist layer 1506, without using a hard mask layer. In this embodiment, the etch stops and mask materials may be selected based on their etching characteristics and characteristics. The materials may also be selected based on mechanical stress characteristics as to maintain the structural integrity of the filter membrane (e.g., no cracking or buckling). The materials may also be selected to minimize warpage of the filter membrane to a level that is acceptable for conventional lithography techniques.

Those having skill in the art will further appreciate that the various illustrative logical blocks, modules, circuits, and process steps described in connection with the implementations disclosed herein may be implemented as electronic hardware, computer software, or combinations of both. To clearly illustrate this interchangeability of hardware and software, various illustrative components, blocks, modules, circuits, and steps have been described above generally in terms of their functionality. Whether such functionality is implemented as hardware or software depends upon the particular application and design constraints imposed on the overall system. Skilled artisans may implement the described functionality in varying ways for each particular application, but such implementation decisions should not be interpreted as causing a departure from the scope of the present embodiments. A person of ordinary skill in the art will appreciate that a portion, or a part, may comprise something less than, or equal to, a whole. For example, a portion of a collection of pixels may refer to a sub-collection of those pixels.

The steps of a method or process described in connection with the implementations disclosed herein may be embodied directly in hardware, in a software module executed by a processor, or in a combination of the two. A software module may reside in RAM memory, flash memory, ROM memory, EPROM memory, EEPROM memory, registers, hard disk, a removable disk, a CD-ROM, or any other form of non-transitory storage medium known in the art. An exemplary computer-readable storage medium is coupled to the processor such the processor can read information from, and write information to, the computer-readable storage medium. In the alternative, the storage medium may be integral to the processor. The processor and the storage medium may reside in an ASIC. The ASIC may reside in a user terminal, camera, or other device. In the alternative, the processor and the storage medium may reside as discrete components in a user terminal, camera, or other device.

Headings are included herein for reference and to aid in locating various sections. These headings are not intended to limit the scope of the concepts described with respect thereto. Such concepts may have applicability throughout the entire specification.

The previous description of the disclosed implementations is provided to enable any person skilled in the art to make or use the embodiments described herein. Various modifications to these implementations will be readily apparent to those skilled in the art, and the generic principles defined herein may be applied to other implementations without departing from the spirit or scope of the embodiments. Accordingly, the disclosed embodiments are not intended to be limited to the implementations shown herein but instead are to be accorded the widest scope consistent with the principles and novel features disclosed herein.

1. A device, comprising:
at least one filter, comprising
a filter structure comprising multiple through holes from a first side of the filter structure to a second side of the filter structure and arranged in a repeating pattern such that each of the through holes is separated from the other through holes by a horizontal pitch and a vertical pitch, each of the through holes having a first opening on the first side of the filter structure, a second opening on the second side of the filter structure, and a passageway through the filter structure between the first opening and the second opening, the passageways of the through holes including one or more sidewalls extending between the first opening to the second opening, the first and second openings sized to capture an object in the through hole, wherein the filter structure has a thickness greater or equal to 1 μm and less than or equal to 20 μm measured along a z-axis of the filter structure; and
a substrate including a plurality of vanes that supports at least a portion of the filter structure, the filter structure disposed relative to the plurality of vanes such that the second side of the filter structure is adjacent to the plurality of vanes.

2. The device of claim 1, wherein the device comprises a microfluidic chip comprising a plurality of filters.

3. The device of claim 2, wherein the plurality of filters are arranged in a grid-like pattern.

4. The device of claim 1, wherein the substrate comprises one or more of polydimethylsiloxane (PDMS), glass, polymethylmethacrylate (PMMA), polyethylene terephthalate (PET) or polycarbonate (PC).

5. The device of claim 1, wherein the filter structure comprises one or more of polydimethylsiloxane (PDMS), glass, polymethylmethacrylate (PMMA), polyethylene terephthalate (PET) or polycarbonate (PC).

6. The device of claim 1, wherein the filter structure comprises silicon oxynitride.

7. The device of claim 1, wherein a size of the first opening size is different than a size of the second opening.

8. The device of claim 1, wherein the passageways of the through holes include one or more sidewalls extending between the first opening to the second opening.

9. The device of claim 8, wherein the one or more sidewalls include at least one tapered sidewall.

10. The device of claim 9, wherein the at least one tapered sidewall is tapered at an angle relative to a line that is generally perpendicular to the first and second side of the filter membrane.

11. The device of claim 10, wherein the at least one tapered sidewall has two or more regions, each of the two or more regions tapered at a different angle relative to a line that is generally perpendicular to the first and second side of the filter membrane.

12. The device of claim 10, wherein the at least one tapered sidewall includes three regions, each of the three regions tapered at a different angle relative to a line that is generally perpendicular to the first and second side of the filter membrane.

13. The device of claim 8, wherein at the one or more sidewalls are curved.

14. The device of claim 1, wherein the first and second openings have a first dimension of between about 4 μm and about 10 μm and a second dimension of between about 4 μm and about 10 μm.

15. The device of claim 1, wherein the through holes are dimensioned to capture and retain a single red blood cell in the through hole.

16. The device of claim 1, wherein the through holes are dimensioned to allow a mature disk-shaped red blood cell to pass through the through hole and capture a single fetal nucleated red blood cell in the through hole.

17. The device of claim 1, wherein the through holes are rectangular shaped.

18. The device of claim 17, wherein the first openings of the through holes have one or more corners that are chamfered or rounded.

19. The device of claim 17, wherein the second openings of the through holes have one or more corners that are chamfered or rounded.

20. The device of claim 1, wherein the through holes are oval-shaped.

21. The device of claim 1, wherein the through holes are circular-shaped.

22. The device of claim 1, wherein a cross-section of the second opening has at least one dimension that is smaller than one dimension of a cross-section of the first opening.

23. The device of claim 1, wherein the first openings and second openings each have a first dimension of between about 4 μm and about 10 μm and a second dimension of between about 4 μm and about 10 μm.

24. The device of claim 1, wherein the through holes have generally circular openings with diameters between about 4 μm and about 10 μm.

25. The device of claim 1, wherein the horizontal pitch is about 20 μm and the vertical pitch is about 10 μm.

26. The device of claim 1, wherein the plurality of vanes are hexagonal-shaped.

27. The device of claim 1, wherein the plurality of vanes are rectangular-shaped.

28. The device of claim 1, wherein the plurality of vanes are square-shaped.

29. The device of claim 1, wherein the vanes has a thickness of about 0.1 millimeter.

30. The device of claim 1, wherein the filter structure is formed on the substrate.

31. The device of claim 1, wherein the filter structure has a thickness in the range of about 1 μm to about 20 μm.

32. The device of claim 1, wherein the filter materials are configured to withstand pressures greater than or equal to 3 psi resulting from fluid flow over and/or though the filter structure.

33. A device, comprising:
at least one filter, comprising
means for capturing cell-sized objects each in one of a plurality of through holes arranged in a repeating pattern; and
means for supporting the means for capturing,
wherein the at least one filter is configured to withstand pressures greater than or equal to 3 psi resulting from fluid flow over and/or though the at least one filter,
wherein the means for capturing comprise a filter structure comprising multiple through holes from a first side of the filter structure to a second side of the filter structure and arranged in a repeating pattern such that each of the through holes is separated from the other through holes by a horizontal pitch and a vertical pitch, each of the through holes having a first opening on the first side of the filter structure, a second opening on the second side of the filter structure, and a passageway through the filter structure between the first opening and the second opening, the passageways of the through holes including one or more sidewalls extending between the first opening to the second opening, the first and second openings sized to capture an object in the through hole;
wherein the means for supporting comprises a substrate including a plurality of vanes that supports at least a portion of the filter structure, the filter structure disposed relative to the plurality of vanes such that the second side of the filter structure is adjacent to the plurality of vanes; and
wherein the through holes are dimensioned to capture and retain a single red blood cell in the through hole.

34. (canceled)

35. The device of claim 33, wherein the one or more sidewalls include at least one tapered sidewall.

36. The device of claim 35, wherein the at least one tapered sidewall is tapered at an angle relative to a line that is generally perpendicular to the first and second side of the filter membrane.

37. The device of claim 35, wherein the at least one tapered sidewall has two or more regions, each of the two or more regions tapered at a different angle relative to a line that is generally perpendicular to the first and second side of the filter membrane.

38. The device of claim 35, wherein the at least one tapered sidewall includes three regions, each of the three regions tapered at a different angle relative to a line that is generally perpendicular to the first and second side of the filter membrane.

39. The device of claim 33, wherein the one or more sidewalls are curved.

40. (canceled)

41. The device of claim 33, wherein the through holes are dimensioned to allow a mature disk-shaped red blood cell to pass through the through hole and capture a single fetal nucleated red blood cell in the through hole.
